(19) United States
(12) Patent Application Publication
(10) Pub. No.: US 2024/0010709 A1
(43) Pub. Date: Jan. 11, 2024

(54) METHODS FOR PREPARING MAMMALIAN CELLS FOR PERFUSION CELL CULTURE

(71) Applicant: Genentech, Inc., South San Francisco, CA (US)

(72) Inventors: Ryan Christopher Hundley, Florence, MT (US); Michael Wilson Laird, Oakland, CA (US); Jerome Joseph Bill, San Mateo, CA (US); Christopher J. Dowd, Belmont, CA (US)

(73) Assignee: Genentech, Inc., South San Francisco, CA (US)

(21) Appl. No.: 18/201,904

(22) Filed: May 25, 2023

Related U.S. Application Data
(60) Provisional application No. 63/345,796, filed on May 25, 2022.

(51) Int. Cl. C07K 16/00; C12P 21/00

(52) U.S. Cl. CPC C07K 16/00; C12P 21/005; C07K 2317/14

(57) ABSTRACT

Methods for preparing mammalian cells for perfusion cell culture processes that improve the growth and productivity of the cells are provided. Also provided are cell culture methods that involve subjecting mammalian cells to one or more perfusion procedures, which involve generating a solids phase and a liquid phase with a continuous flow centrifuge, and utilizing at least a portion of the solids phase to sustain, maintain and/or initiate a new cell culture. Perfusion bioreactor systems and components thereof are also provided.

The present application claims priority to U.S. Provisional Patent Application No. 63/345,796, filed May 25, 2022, the contents of which are hereby incorporated by reference in its entirety.

### FIELD OF THE INVENTION

The present disclosure generally relates to methods for preparing mammalian cells for perfusion cell culture processes that improve the growth and productivity of the cells. More specifically, the disclosure relates to improved cell culture methods wherein mammalian cells are subjected to one or more perfusion procedures that involve generating a solids phase and a liquid phase with a continuous flow centrifuge, and utilizing at least a portion of the solids phase to sustain, maintain, and/or initiate a new cell culture. The present disclosure also relates to perfusion bioreactor systems and components thereof.

### BACKGROUND OF THE INVENTION

Despite significant strides in the field over the last several decades, biomanufacturing processes involving industrial-scale culture of mammalian cells and the production of products therein remain fraught with significant challenges. For example, typical bioreactor systems that are used for such processes must maintain a sterile, closed environment, making the addition of fresh cell culture medium and the removal of spent cell culture medium problematic. As such, cells that are grown under batch or fed-batch process conditions often face nutrient limitations when their cell culture medium is depleted of vital components. In addition, the accumulation of unwanted metabolic byproducts in the cell culture can further limit growth and potentially be damaging to the desired product. As such, there remains a strong need for improved cell culture systems and methods that can increase growth and productivity by addressing these, and other, limitations.

As compared to bacterial cell cultures, mammalian cell cultures typically have lower production rates and generate lower production yields. Thus, a significant quantity of research focuses on mammalian cell culture conditions and methods that can optimize the polypeptide output, i.e., conditions and methods that support high cell density and high titer of protein. For example, it has been determined that restricted feeding of glucose to mammalian cell cultures in fed-batch processes controls lactate production without requiring constant-rate feeding of glucose (see, e.g., U.S. Patent Application Publication No. US20050070013).

Two cell culture processes are primarily used for large-scale protein production: the fed-batch process and the perfusion process. The primary goal of these methods is to add nutrients, e.g., glucose, as they are being consumed by the cells, and to remove metabolic waste products, e.g., lactic acid and ammonia, as they are being produced. In a fed-batch process, cells typically receive inoculation medium containing glucose at the initiation of the culture and at one or more time points after initiation, but before termination, of the culture. While this approach can help control lactic acid production by cultured cells at relatively low levels, maximum cell density, growth rate, and cell viability levels are not achieved due to glucose limiting conditions. Consequently, the number of cells and/or quantity of product produced by the cells is not maximized.

In a perfusion process, cells also receive inoculation base medium, and at the point when cells achieve a desired cell density, cell perfusion is initiated such that the spent medium is replaced by fresh medium. The perfusion process allows the culture to achieve higher cell density, and thus enables the production of a large quantity of cells and/or product. However, at larger, industrially-relevant production scales, perfusion processes require very large quantities of fresh cell culture medium. Moreover, any product that is secreted by the cells into the culture medium is lost when spent medium is removed. This requires separate harvest steps to capture product in the spent medium, or else an overall loss of efficiency if the spent medium is discarded.

Thus, there exists a need for improved methods of large-scale cell culture that can maximize cell viability, cell concentration, and the quantity of protein produced, as well as minimize product losses in spent culture medium. The present disclosure addresses these and other needs.

### SUMMARY OF THE INVENTION

Provided herein are cell culturing methods that comprise conducting a perfusion procedure on a cell culture using a continuous flow centrifuge to generate a solids phase and a liquid phase, and returning at least a portion of the solids phase and a volume of cell culture medium to a culture vessel to sustain, maintain, and/or initiate a new cell culture. In some embodiments, the methods can be used to achieve a perfusion rate that ranges from about 0.5 up to about 6 or more vessel volumes per day (VVD). The continuous flow centrifuge is used as part of cell culturing, that is, as part of the “upstream” process for production of, e.g., therapeutic polypeptides or proteins such as antibodies or fusion proteins, as opposed to the use of a continuous flow centrifuge as part of clarification as a part of a “downstream” purification of such therapeutic polypeptides or proteins the cell culture has produced.

In a first aspect, presented herein is a method for culturing mammalian cells, e.g., mammalian cells that have been engineered to produce a protein, e.g., a therapeutic protein such as an antibody, prior to clarification in preparation for protein purification; that is, in a pre-production vessel, e.g., bioreactor. In specific embodiments, the method comprises placing a plurality of mammalian cells and a volume of culture medium in a culture vessel to generate a cell culture. In other specific embodiments, the method comprises culturing the cell culture to a cell density of greater than 1% packed cell volume (PCV). In specific embodiments, the method includes conducting a perfusion procedure on the cell culture. In more specific embodiments, the perfusion procedure comprises transferring at least a portion of the cell culture to a continuous flow centrifuge. In more specific embodiments, the perfusion procedure comprises operating the continuous flow centrifuge to generate a solids phase having a cell density of greater than or equal to 1% PCV. In more specific embodiments, the perfusion procedure comprises returning the solids phase and a volume of cell culture medium to the culture vessel to achieve a perfusion rate that ranges from 0.5 to 6 vessel volumes per day (VVD). In a specific embodiment, a low perfusion rate of about 0.7 VVD is used, and/or a high perfusion rate of about 4 to about 5 VVD is used.

In specific embodiments, following completion of the perfusion procedure, the cell culture may have a cell density of 0.2% PCV or greater for, e.g., 0.2% to about 30% PCV.

In certain embodiments, the perfusion procedure comprises increasing or decreasing the perfusion rate in a constant manner. In certain embodiments, the perfusion procedure comprises increasing or decreasing the perfusion rate in a variable manner.

In certain embodiments, the time period over which a perfusion procedure, or a cell culture method comprising such perfusion procedure, is conducted ranges from 0.5 hours up to about 5 hours, such as about 1, 1.5, 2, 2.5, 3, 3.5, 4 or 4.5 hours or more. In certain embodiments, a time period over which a perfusion procedure, or a cell culture method comprising such perfusion procedure, is conducted ranges from about 5 hours up to about 24 hours, such as about 6, 7, 8, 9, 10, 11, 12, 13, 14, 15, 16, 17, 18, 19, 20, 21, 22, or 23 hours. In certain embodiments, a time period over which a perfusion procedure, or a cell culture method comprising such perfusion procedure, is conducted ranges from about 1 day up to about 20 days, such as about 2, 3, 4, 5, 6, 7, 8, 9, 10, 11, 12, 13, 14, 15, 16, 17, 18 or 19 days. In other embodiments, a perfusion procedure, or the cell culture method comprising the perfusion procedure, is conducted up to 20, 25, 30, 35, 40, 45, 50, 55, 60, 65, 70, 75, 80, 85 or 90 days. Moreover, perfusion procedures in accordance with embodiments above may be conducted in a semi-continuous, discontinuous or “punctuated” manner, wherein a perfusion procedure is conducted, for example, once per day over a period of several days.

In certain embodiments, the continuous flow centrifuge comprises a disc stack bowl. In certain embodiments, the continuous flow centrifuge comprises a tubular bowl. In certain embodiments, the continuous flow centrifuge may have an operating speed that ranges from 3,000 to 10,000 revolutions per minute (RPM). In certain embodiments, the continuous flow centrifuge includes a sterilizable component. In certain embodiments, the continuous flow centrifuge includes a disposable component. In certain embodiments, the continuous flow centrifuge may have a sigma value that ranges from 1,000 m² to 200,000 m². In certain embodiments, following completion of the centrifugation procedure, the cell culture may have a viability percentage that is greater than or equal to 40%, 45%, 50%, 55%, 60%, 65%, 70%, 75%, 80%, or 85%, e.g., 85%, 86%, 87%, 88%, 89%, 90%, 91%, 92%, 93%, 94%, 95%, 96%, 97% or 98%. In certain embodiments, the cell culture maintains a viability percentage that may be greater than or equal to 40%, 45%, 50%, 55%, 60%, 65%, 70%, 75%, 80%, or 85%, e.g., 85%, 86%, 87%, 88%, 89%, 90%, 91%, 92%, 93%, 94%, 95%, 96%, 97% or 98% over the time period of 1 to 90 days, e.g., for, or up to, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85 or 90 days. In certain embodiments, the mammalian cells include recombinant mammalian cells. In certain embodiments, the recombinant mammalian cells include recombinant Chinese hamster ovary (CHO) cells. In certain embodiments, the cell culture may have a lactate concentration that is less than or equal to 4 g/L. In certain embodiments, the recombinant mammalian cells may produce a secreted product. In certain embodiments, the secreted product includes a recombinant protein. In certain embodiments, the recombinant protein may be an antibody. In certain embodiments, the culture vessel may have a working volume that is greater than or equal to 80 L. In certain embodiments, the culture vessel may have a total volume that ranges from 100 L to 3,000 L. In certain embodiments, the culture vessel may have a total volume of 100 L. In certain embodiments, the culture vessel may have a total volume of 3,000 L. In certain embodiments, a culture vessel has a total volume that ranges from 100 L to 30,000 L. In certain embodiments, a culture vessel has a total volume that ranges from 5,000 L to 30,000 L. In certain embodiments, a culture vessel has a total volume that ranges from 100 L to 6,000 L. In some embodiments, the method may include transferring at least a portion of the cell culture to a different culture vessel to initiate a second cell culture. In certain embodiments, the second cell culture has an initial cell density that ranges from greater than or equal to 0.1% to 10% PCV. In some embodiments, the method may comprise transferring at least a portion of the cell culture to a production culture vessel to initiate a production culture having a starting cell density that ranges from greater than or equal to 0.10% to 10% PCV. In some embodiments, the method may comprise culturing the production culture under batch or fed-batch process conditions. In some embodiments, the method may comprise isolating a liquid phase from the continuous flow centrifuge and performing a purification procedure on a secreted product therein. In some embodiments, the method may include adding fresh culture fluid (e.g., cell culture medium and/or cell culture media) to the culture vessel.

In certain embodiments, the cell culture methods provided herein comprise generating a culture of mammalian cells having a cell density of greater than or equal to 0.1% PCV. In certain embodiments, the method includes placing a plurality of mammalian cells and a volume of cell culture medium in a culture vessel to generate a cell culture. In certain embodiments, the method comprises culturing the cell culture to a cell density of greater than or equal to 10% PCV, e.g., about 12%, 14%, 16%, 18%, 20%, 22%, 24%, 26%, 28% or about 30%. In certain embodiments, the method comprises conducting a perfusion procedure on the cell culture. In certain embodiments, the perfusion procedure comprises transferring at least a portion of the cell culture to a continuous flow centrifuge. In certain embodiments, the perfusion procedure comprises operating the continuous flow centrifuge to generate a solids phase having a cell density that ranges from about 1% to about 10%, about 20%, about 30%, about 40% or about 50% PCV. In specific embodiments, the cell density ranges from about 20% to about 40%. In more specific embodiments, the cell density ranges from about 30% to about 35%. In certain embodiments, the perfusion procedure comprises returning a portion of the solids phase and a volume of cell culture medium to the culture vessel to achieve a perfusion rate that ranges from 0.5 to 6 VVD. In certain embodiments, following completion of the perfusion procedure, the cell culture density increases, or has increased, by greater than 0.1% PCV, e.g., greater than or equal to 1% PCV or greater than or equal to 10% PCV. In specific embodiments, the solids phase cell density ranges from about 20% PCV to about 40% PCV.

In certain embodiments, the cell culture methods provided herein comprise generating a culture of mammalian cells comprising at least 4.8×10¹² cells. In specific embodiments, the method comprises placing a plurality of mammalian cells and a volume of cell culture medium in a culture vessel having a working volume of at least, no more than, or about 80 L to generate a cell culture having a starting cell density of greater than or equal to 1 million cells/mL. In specific embodiments, the method comprises culturing the cell culture to a cell density of greater than or equal to 1% PCV, or greater than or equal to 10% PCV. In specific embodiments, the method comprises conducting a perfusion procedure on the cell culture. In specific embodiments, the perfusion procedure comprises transferring at least a portion of the cell culture to a continuous flow centrifuge comprising a disposable disc stack bowl and comprising a sigma factor that ranges from 1,000 to 200,000 m². In specific embodiments, the method comprises operating the continuous flow centrifuge to generate a solids phase having a cell density of greater than or equal to 1% PCV, to e.g., about 10%, about 20%, about 30%, about 40% or about 50% PCV. In specific embodiments, the method comprises returning at least a portion of the solids phase and a volume of cell culture medium to the culture vessel to achieve a perfusion rate that ranges from 0.5 to 6 VVD. In specific embodiments, following completion of the perfusion procedure, the cell culture has a cell density of greater than or equal to 60 million cells/mL.

In certain embodiments, provided herein are methods of generating an inoculation culture of mammalian cells comprising at least 2.16×10¹⁴ cells in total, or about 1.5-2.5×10⁶ cells/mL. In specific embodiments, the method comprises placing a plurality of mammalian cells and a volume of cell culture medium in a culture vessel having a working volume of up to, at least, or about 3,000 L or 3,600 L to generate a cell culture having a starting cell density of greater than or equal to 1 million cells/mL (1×10⁶ cells/mL). In specific embodiments, the method comprises culturing the cell culture to a cell density of greater than or equal to 10% PCV. In specific embodiments, the method comprises conducting a perfusion procedure on the cell culture. In specific embodiments, the perfusion procedure comprises transferring at least a portion of the cell culture to a continuous flow centrifuge comprising a disposable disc stack bowl, and further comprising a sigma factor that ranges from 1,000 to 200,000 m². In specific embodiments of the methods provided herein, the perfusion procedure includes operating the continuous flow centrifuge to generate a solids phase having a cell density of greater than or equal to 1% PCV, e.g., up to about 10%, about 20%, about 30%, about 40% or about 50% PCV. In certain embodiments, the perfusion procedure includes returning at least a portion of the solids phase and a volume of cell culture medium to the culture vessel to achieve a perfusion rate that ranges from 0.5 to 6 VVD. In certain embodiments, following completion of the perfusion procedure, the cell culture has a cell density of greater than or equal to 60×10⁶ cells/mL.

In certain embodiments, the cell culture produced by the methods described herein is part of a seed train in pre-production cell culturing. For example, a cell culture produced by the methods described herein may be used to seed another, larger, bioreactor that constitutes a further step in pre-production cell culture. In specific embodiments, the cells in said cell culture are concentrated using a centrifuge, e.g., a continuous flow centrifuge, and the resulting concentrated cells (that is, the solids phase or heavy phase) are used to initiate another cell culture vessel, e.g., bioreactor. In certain embodiments, the bioreactor is a pre-production bioreactor. In certain other embodiments, the bioreactor is a production bioreactor. In specific embodiments, the solids phase or heavy phase is used to initiate more than one, e.g., 2, 3 or more other cell cultures in 2, 3 or more cell culture vessels, e.g., bioreactors. In certain specific embodiments, the heavy phase or solids phase is used to initiate 2 or 3 production cell cultures.

These and further aspects will be further explained in the rest of the disclosure, including the examples.

### BRIEF DESCRIPTION OF THE DRAWINGS

FIG. 1 is schematic diagram of a system for perfusion bioprocessing in accordance with one or more embodiments.

FIG. 2 is a flowchart of a process for culturing mammalian cells in accordance with one or more embodiments.

FIG. 3 is a flowchart of a process for culturing mammalian cells in accordance with one or more embodiments.

FIG. 4 is a flowchart of a process for generating an inoculation culture of mammalian cells having a cell density of greater than 0.10% PCV in accordance with one or more embodiments.

FIG. 5 is a flowchart of a process for generating an inoculation culture of mammalian cells comprising at least 4.8×10¹² cells in accordance with one or more embodiments.

FIG. 6 is a flowchart of a process for generating an inoculation culture of mammalian cells comprising at least 2.16×10¹⁴ cells in accordance with one or more embodiments.

### DETAILED DESCRIPTION OF THE PREFERRED EMBODIMENTS

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

Where a range of values is provided, it is understood that each intervening value, to the tenth of the unit of the lower limit unless the context clearly dictates otherwise, between the upper and lower limit of that range and any other stated or intervening value in that stated range is encompassed within the described embodiments. The upper and lower limits of these smaller ranges may independently be included in the smaller ranges is also encompassed within the described embodiments, subject to any specifically excluded limit in the stated range. Where the stated range includes one or both of the limits, ranges excluding either or both of those included limits are also included in the described embodiments.

In the following description, numerous specific details are set forth to provide a more thorough understanding of the technologies described herein. However, it will be apparent to one of skill in the art that the present technologies described herein may be practiced without one or more of these specific details. In other instances, well-known features and procedures well known to those skilled in the art have not been described in order to avoid obscuring the present description of the various embodiments.

All references cited throughout the disclosure, including patent applications and publications, are incorporated by reference herein in their entirety.

### I. DEFINITIONS

By “comprising” it is meant that the recited elements are required in the composition/method/kit, but other elements may be included to form the composition/method/kit etc. within the scope of the claim.

By “consisting essentially of”, it is meant a limitation of the scope of composition or method described to the specified materials or steps that do not materially affect the basic and novel characteristic(s) of the described embodiment.

By “consisting of”, it is meant the exclusion from the composition, method, or kit of any element, step, or ingredient not specified in the claim.

The terms “culture” and “cell culture” as used interchangeably herein refer to a cell population that is suspended in a cell culture medium under conditions that are suitable for survival and/or growth of the cell population. As used herein, these terms may refer to the combination comprising the cell population (e.g., the animal cell culture) and the medium in which the population is suspended.

The terms “medium”, “cell culture medium”, and “culture medium” are used interchangeably herein to refer to a solution containing nutrients that nourish cells, e.g., mammalian cells, and can also refer to medium in combination with cells. The term “inoculation medium” refers to the medium that is used to generate an inoculation cell culture. The term “production medium” refers to the medium that is used to generate a production cell culture.

The term “batch culture” as used herein refers to a method of culturing cells in which all the components that will ultimately be used in culturing the cells, including the cell culture medium as well as the cells themselves, are provided at the beginning of the culturing process. A batch culture is typically stopped at a designated time point, and the cells and/or components in the medium are harvested and optionally purified.

The term “fed-batch culture” as used herein refers to a method of culturing cells in which additional components are provided to the culture at some time subsequent to the beginning of the culture process. The provided components typically comprise nutritional supplements for the cells that have been depleted during the culturing process. A fed-batch culture is typically stopped at a designated time point, and the cells and/or components in the medium are harvested and optionally purified.

The term “perfusion culture” as used herein refers to a method of culturing cells in which additional fresh medium is added to the culture (subsequent to the beginning of the culture process), either continuously over some time period or intermittently over some time period, and, simultaneously, spent culture medium is removed. The fresh medium typically provides nutritional supplements for the cells that have exhausted previously existing nutritional supplements during the culturing process. A cell culture product, such as a protein (e.g., an antibody), which may be present in the spent medium, is optionally purified. Perfusion also allows for removal of unwanted cellular waste products (e.g., excess metabolites, such as lactate) from the cell culture growing in the bioreactor. Perfusion culture can be integrated into continuous flow bioprocesses workflows for constant product production and/or purification.

The term “perfusion rate” as used herein refers to the rate at which cell culture medium is removed for a perfusion culture and replaced with fresh cell culture medium (e.g., medium exchange rate). In some embodiments, perfusion rate is measured in vessel volumes per day, or VVD. In some embodiments, perfusion rate is measured in units of volume per unit time, such as liters per minute (LPM), etc.

The terms “bioreactor” or “fermenter” or “vessel” or “culture vessel” as used herein refer to any vessel used for the growth of a prokaryotic or eukaryotic cell culture, e.g., an animal cell culture (e.g., a mammalian cell culture). When the term “Vessel Volumes per Day (VVD)” is used herein to describe cell culture perfusion flow rates, for example, the “vessel” can be a bioreactor, a single-use bioreactor, a fermenter or culture vessel. A bioreactor can be of any size, provided it is useful for the culturing of cells. Typically, a bioreactor will be at least 100 mL and may be at least about 1, 10, 20, 80, 100, 250, 300, 350, 400, 450, 500, 1,000, 2,000, 2,500, 3,000, 3,500, 4,000, 4,500, 5,000, 7,500, 8,000, 10,000, 12,000, 15,000, 16,000, 18,000, 20,000, 22,000, 24,000, 26,000, 28,000 or 30,000 liters or more, or any intermediate volume. A production may be from 20 liters to 80 liters, from 80 liters to 100 liters, from 100 liters to 250 liters, from 250 liters to 300 liters, from 300 liters to 350 liters, from 350 liters to 400 liters, 400 liters to 450 liters, 450 liters to 500 liters, 500 liters to 1,000 liters, 1000 liters to 2,000, 2000 liters to 2,500 liters, 2500 liters to 3,000 liters, 3000 liters to 3,500 liters, 3500 liters to 4,000 liters, 4000 liters to 4,500 liters, 4500 liters to 5,000 liters, 5000 liters to 7,500 liters, 7000 liters to 8,000 liters, 8000 liters to 10,000 liters, 10,000 liters to 12,000 liters, 12,000 liters to 15,000 liters, 15,000 liters to 16,000, 16,000 liters to 18,000 liters, 18,000 liters to 20,000 liters, 20,000 liters to 22,000 liters, 20,000 liters to 24,000 liters, 24,000 liters to 26,000 liters, 26,000 liters to 28,000 liters, or 28,000 liters to 30,000 liters in volume. The internal conditions of the bioreactor, including, but not limited to, dissolved oxygen (dO₂), pH, and temperature, agitation, foaming, and pressure are typically controlled during the culturing period.

The terms “inoculation culture” and “inoculation cell culture” as used interchangeably herein refer to a cell culture that is used primarily for generating cell mass (i.e., increasing the number of viable cells in the culture) in order to reach a target cell density that can be used to initiate a larger volume cell culture (e.g., a larger inoculation cell culture, or a production cell culture). In some embodiments, an inoculation cell culture can be transferred in its entirety into a production bioreactor and combined with production cell culture medium in order to initiate a production cell culture. In some embodiments, an inoculation cell culture can be transferred in its entirety into a larger inoculation cell culture bioreactor and combined with additional inoculation cell culture medium in order to initiate an inoculation cell culture having a larger volume. In some embodiments, a first portion of an inoculation culture can be transferred into a production bioreactor and combined with production cell culture medium to initiate a production cell culture, and a second portion of the inoculation culture can be combined with inoculation culture medium to initiate a new inoculation cell culture. In general, one or more inoculation cell cultures that are grown before initiation of a production cell culture are referred to as “pre-production cell cultures”.

The term “inoculation bioreactor” as used herein refers to a bioreactor that is used to contain an inoculation cell culture. Inoculation bioreactors are generally used in the production of cell mass, and generally have a lower volume than a production bioreactor. The volume of a large-scale cell culture inoculation bioreactor is generally greater than about 100 mL, typically at least about 10 liters, and may be 20, 50, 80, 100, 250, 300, 350, 400, 450, 500, 1,000, 2,000, 2,500, 3,000, 3,500, 4,000, 4,500, or 5,000 liters or more, or any intermediate volume.

The terms “production culture” and “production cell culture” as used interchangeably herein refer to a cell culture that is used primarily for generating a product, and which generally represents the last or final step in a cell culture process. In some embodiments, a production culture can be used to generate cells as the product. In some embodiments, a production culture can be used to generate a product within the cells that are cultured in the production culture. In some embodiments, a production culture can be used to generate a product that is secreted by the cells that are cultured in the production culture.

The term “production bioreactor” as used herein refers to a bioreactor that is used to contain a production cell culture. Production bioreactors are generally used in the production of a cell or protein product of interest. The volume of a large-scale cell culture production bioreactor is generally greater than about 100 mL, typically at least about 10 liters, and may be 20, 80, 100, 250, 300, 350, 400, 450, 500, 1,000, 2,000, 2,500, 3,000, 3,500, 4,000, 4,500, 5,000, 7,500, 8,000, 10,000, 12,000, 15,000, 16,000, 18,000, 20,000, 22,000, 24,000, 26,000, 28,000 or 30,000 liters or more, or any intermediate volume. A production bioreactor may be from 20 liters to 80 liters, from 80 liters to 100 liters, from 100 liters to 250 liters, from 250 liters to 300 liters, from 300 liters to 350 liters, from 350 liters to 400 liters, 400 liters to 450 liters, 450 liters to 500 liters, 500 liters to 1,000 liters, 1000 liters to 2,000, 2000 liters to 2,500 liters, 2500 liters to 3,000 liters, 3000 liters to 3,500 liters, 3500 liters to 4,000 liters, 4000 liters to 4,500 liters, 4500 liters to 5,000 liters, 5000 liters to 7,500 liters, 7000 liters to 8,000 liters, 8000 liters to 10,000 liters, 10,000 liters to 12,000 liters, 12,000 liters to 15,000 liters, 15,000 liters to 16,000, 16,000 liters to 18,000 liters, 18,000 liters to 20,000 liters, 20,000 liters to 22,000 liters, 20,000 liters to 24,000 liters, 24,000 liters to 26,000 liters, 26,000 liters to 28,000 liters, or 28,000 liters to 30,000 liters in volume.

A suitable bioreactor (e.g., an inoculation bioreactor or a production bioreactor) may include (i.e., constructed of) any material that is suitable for holding cells suspended in media under the desired culture conditions, and is conducive to cell growth, maintained cellular viability, and/or production of a product, including glass, plastic or metal. Generally, the material(s) should not interfere with expression or stability of a product produced in and/or secreted by the cultured cells, e.g., a protein product. One of ordinary skill in the art will be readily able to choose suitable bioreactors and operating conditions thereof for use in practicing the methods described herein.

The term “cell density” as used herein refers to the number of cells present in a given volume of medium. The term “viable cell density” as used herein refers to the number of live (viable) cells present in a given volume of medium. Various sensors or probes may be incorporated into cell culture systems to directly measure conditions within a cell culture vessel. For example, optical probes may be used to monitor cell growth, however they may not be able to differentiate between live and dead cells. Radio-frequency impedance (RFI) probes may be used to monitor cell growth and may also determine viable cell density by detecting live cells through capacitance measurements. Samples of cells may be obtained from the culture at one or more times during cell culture and the percent viability determined by use of spectroscopy, e.g., Raman spectroscopy, Alamar Blue dye staining, MTT (3-(4,5-dimethylthiazol-2-yl)-2,5-diphenyltetrazolium bromide) assay, Trypan Blue staining, electronic particle counting, EdU assay, XTT assay, WST-1 assay, luminescent ATP assay, and the like. The number of viable cells per mL can then be determined.

The term “packed cell volume” or “PCV” as used herein refers to the ratio of the volume of a sample of cell culture fluid that is occupied by packed cells after the sample of cell culture fluid has been centrifuged, to the volume of the sample of cell culture fluid. As with cell density, optical and RFI probes may be used to determine PCV.

The term “cell viability” as used herein refers to the ability of cells in culture to survive under a given set of culture conditions or experimental variations. The term as used herein also refers to the portion of the cells that are alive at a particular time in relation to the total number of cells, living and dead, in the culture at that time. Radio-frequency impedance (RFI) probes may be used to monitor cell growth and may also determine viable cell density by detecting live cells through capacitance measurements. Samples of cells may be obtained from the culture at one or more times during cell culture and the percent viability determined by use of Alamar Blue dye staining, MTT (3-(4,5-dimethylthiazol-2-yl)-2,5-diphenyltetrazolium bromide) assay, Trypan Blue staining, electronic particle counting, EdU assay, XTT assay, WST-1 assay, luminescent ATP assay, and the like.

As used herein, the terms “polypeptide” and “polypeptide product” are synonymous with the terms “protein” and “protein product”, respectively, and as is generally understood in the art, refer to at least one chain of amino acids linked via sequential peptide bonds. In certain embodiments, a “protein of interest” or a “polypeptide of interest”, or the like, is a protein encoded by an exogenous nucleic acid molecule that has been transformed into a host cell. In certain embodiments, wherein an exogenous DNA molecule with which the host cell has been transformed encodes a “protein of interest”, the nucleic acid sequence of the exogenous DNA determines the sequence of amino acids. In certain embodiments, a “protein of interest” is a protein encoded by a nucleic acid molecule that is endogenous to the host cell. In certain embodiments, expression of such an endogenous protein of interest is altered by transfecting a host cell with an exogenous nucleic acid molecule that may, for example, contain one or more regulatory sequences and/or encode a protein that enhances expression of the protein of interest.

The term “titer” as used herein refers to the total amount of a product (e.g., polypeptide) of interest produced by a cell culture (e.g., an animal cell culture), divided by a given volume of culture medium. As such, “titer” refers to a concentration of the polypeptide of interest. Titer is typically expressed in units of grams or milligrams of polypeptide per milliliter or liter of medium.

An “isolated” product (e.g., an antibody) is one which has been identified and separated and/or recovered from a component of its natural environment. Contaminant components of its natural environment are materials which would interfere with diagnostic or therapeutic uses for the product, and may include enzymes, hormones, and other proteinaceous or non-proteinaceous solutes. In preferred embodiments, the product will be purified (1) to greater than 95% by weight of product as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. An isolated product includes the product in situ within recombinant cells, since at least one component of the product's natural environment will not be present. Ordinarily, however, isolated product will be prepared by at least one purification step.

The terms “sigma value” and “sigma factor” as used interchangeably herein in reference to a centrifuge refer to the operation constant that represents the geometry and speed ofthe centrifuge. A sigma factor can be used to compare two or more centrifuges having different sizes, geometries, and/or operating speeds.

The term “solids phase” in the context of the cell culture method described herein refers to the flow from a continuous centrifuge that comprises the bulk of the cells that are being separated from the culture medium (“liquid phase”, below).

The term “liquid phase” in the context of the cell culture method described herein refers to the flow from a continuous centrifuge that comprises predominantly liquid, e.g., cell culture medium, and few to no cells.

The terms “solids phase to liquid phase ratio” and “split ratio”, as used interchangeably herein, refer to the ratio of a volume of solids phase to a volume of liquid phase that are combined to sustain, maintain and/or initiate a new cell culture. For example, the centrifuge may be adjusted to volumetrically flow 50% of the centrifuge inlet flow to the solids outlet and 50% of the centrifuge inlet flow through the liquid outlet for a split ratio of 50:50 solids to liquid. In the same manner, a volumetric flow of 75% of the centrifuge inlet flow to the solids outlet and 25% of the centrifuge inlet flow through the liquid outlet for a split ratio of 75:25, and a volumetric flow of 90% of the centrifuge inlet flow to the solids outlet and 10% of the centrifuge inlet flow through the liquid outlet for a split ratio of 90:10.

The term “residence time”, as used herein, means the time, e.g., the time during perfusion centrifugation, that the cells are outside of the cell culture vessel. Residence time includes time cells are within the centrifuge, and the time cells are in transit between the cell culture vessel and centrifuge.

### II. DETAILED DESCRIPTION

Aspects of the disclosure include methods that involve conducting a perfusion procedure on a cell culture using a continuous flow centrifuge to generate a solids phase and a liquid phase, and returning at least a portion of the solids phase and a volume of cell culture medium to a culture vessel to sustain, maintain and/or initiate a new cell culture. In some embodiments, the methods optionally include returning a portion of the liquid phase of the original cell culture to the new cell culture.

Mammalian cell cultures generally consist of a growth phase, during which the cells grow to a target density (e.g., a target cell concentration or packed cell volume) and a production phase, during which the cells grow less, but produce larger quantities of product (e.g., a recombinant protein) than was produced by the cell culture during the growth phase.

During the growth phase of a cell culture, cells are first mixed with a medium (i.e., inoculation medium) to form a cell culture. Typically, cell culture media provides, without limitation, essential and nonessential amino acids, vitamins, energy sources, lipids, and trace elements required by the cells for at least minimal growth and/or survival. A culture medium may also contain components that enhance growth and/or survival above the minimal rate, including hormones and growth factors. The culture medium is preferably formulated to a pH and salt concentration that is optimal for cell survival and proliferation. In at least one embodiment, the culture medium is a defined culture medium. Defined media are media in which all components have a known chemical structure. In other embodiments, the culture medium may contain an amino acid(s) derived from any source or method known in the art, including, but not limited to, an amino acid(s) derived either from single amino acid addition(s) or from a peptone or protein hydrolysate addition(s) (including animal or plant source(s)). In yet other embodiments, the culture medium used during the cell growth phase may contain concentrated medium, i.e., medium that contains higher concentration of nutrients than is normally necessary and normally provided to a growing culture. One skilled in the art will recognize which cell media, inoculation media, etc. is appropriate for use with a cell culture of a particular cell type, e.g., animal cell (e.g., CHO cells), and the amount of glucose and other nutrients (e.g., glutamine, iron, trace elements) or agents designed to control other culture variables (e.g., the amount of foaming, osmolality) that the medium should contain (see, e.g., Mather, J. P., et al. (1999) “Culture media, animal cells, large scale production,” Encyclopedia of Bioprocess Technology: Fermentation, Bio catalysis, and Bio separation, Vol. 2:777-85; U.S. Patent Application Publication No. US2006/0121568; each of which are hereby incorporated by reference herein in their entireties). The embodiments described herein contemplate variants of such known media, including, e.g., nutrient-enriched variants of such media.

One skilled in the art will also recognize at what temperature and/or concentration a particular cell line should be cultured. For example, most mammalian cells, e.g., CHO cells, grow well within the range of about 35° C. to 39° C., preferably at 37° C., whereas insect cells are typically cultured at 27° C.

Methods in accordance with embodiments of the disclosure can make use of any suitable recombinant host cells, e.g., prokaryotic or eukaryotic host cells, i.e., cells transfected with an expression construct containing a nucleic acid that encodes a polypeptide of interest (e.g., an antibody). A number of mammalian cell lines are suitable host cells for recombinant expression of polypeptides of interest. Mammalian host cell lines include, for example and without limitation, COS, PER.C6, TM4, VERO076, MDCK, BRL-3A, W138, Hep G2, MMT, MRC 5, FS4, CHO, 293T, A431, 3T3, CV-I, C3H10T1/2, Colo205, 293, HeLa, L cells, BHK, HL-60, FRhL-2, U937, HaK, Jurkat cells, Rat2, BaF3, 32D, FDCP-I, PC12, Mix, murine myelomas (e.g., SP2/0 and NSO) and C2C12 cells, as well as transformed primate cell lines, hybridomas, normal diploid cells, and cell strains derived from in vitro culture of primary tissue and primary explants. Any eukaryotic cell that is capable of expressing a product of interest may be used in connection with embodiments of the presently described embodiments. Numerous cell lines are available from commercial sources such as the American Type Culture Collection (ATCC). In various embodiment, a cell culture employs hybridoma cells. In various embodiment, a cell culture employs CHO cells.

In specific embodiments, the cell culture methods provided herein may be used to culture cells producing any kind of therapeutic polypeptide. In certain embodiments, the therapeutic polypeptide is a fusion protein, e.g., a fusion protein comprising an antibody Fc portion or human serum albumin. In other specific embodiments, the therapeutic polypeptide is an antibody or antibody fragment, e.g., a monoclonal antibody, monospecific antibody, bispecific antibody (with or without common light chain), bispecific T cell engager (BiTE), bispecific (mab)₂ antibodies; bispecific F(mab)₂ antibody; single-domain bispecific diabody (scBsDb), single-chain bispecific tandem variable domain (scBsTaFv), trispecific NK cell engager therapy (TriNKET), dual-affinity re-targeting protein (DART), bispecific diabody, tandem diabody (TandAb), half-antibodies (e.g., antibodies comprising an Fc portion but only one CH-VH:CL-VL pair), trifab contorsbody (as described in US Patent Application Publication No. WO 2019/086395; quadroma, scFv, dock-and-lock trivalent fab (DNL-(Fab)₃, single-domain antibody, bispecific single-domain antibody, or the like.

In specific embodiments, the therapeutic polypeptide is atezolizumab, abagovomab, abciximab, abituzumab, abrezekimab, abrilumab, actoxumab, adalimumab, adecatumumab, aducanumab, afasevikumab, afelimomab, alacizumab, alemtuzumab, alirocumab, altumomab pentetate, amatuximab, amivantamab, anatumomab, andecaliximab, anetumab, anifrolumab, ansuvimab, anrukinzumab, apolizumab, aprutumab, ascrinvacumab, aselizumab, atezolizumab, atidortoxumab, atinumab, atoltivimab, atorolimumab, avelumab, azintuxizumab, bamlanivimab, bapineuzumab, basiliximab, bavituximab, bebtelovimab, bectumomab, begelomab, belantamab, belimumab, bemarituzumab, benralizumab, berlimatoxumab, bermekimab, bersanlimab, bertilimumab, besilesomab, bevacizumab, bezlotoxumab, biciromab, bimagrumab, bimekizumab, birtamimab, bivatuzumab, bleselumab, blinatumomab, blontuvetmab, blosozumab, bococizumab, brazikumab, brentuximab, briakinumab, brodalumab, brolucizumab, brontictuzumab, burosumab, cabiralizumab, camidanlumab, camrelizumab, canakinumab, cantuzumab, cantuzumab, caplacizumab, casirivimab, capromab, carlumab, carotuximab, catumaxomab, cedelizumab, cemiplimab, cergutuzumab, certolizumab, cetrelimab, cetuximab, cibisatamab, cilgavimab, cirmtuzumab, citatuzumab, cixutumumab, clazakizumab, clenoliximab, clivatuzumab, codrituzumab, cofetuzumab, coltuximab, conatumumab, concizumab, cosfroviximab, crenezumab, crizanlizumab, crotedumab, cusatuzumab, dacetuzumab, daclizumab, dalotuzumab, dapirolizumab, daratumumab, dectrekumab, demcizumab, denintuzumab, denosumab, depatuxizumab, derlotuximab, detumomab, dezamizumab, dinutuximab, dinutuximab, diridavumab, domagrozumab, dorlimomab, dostarlimab, drozitumab, duligotuzumab, dupilumab, durvalumab, dusigitumab, duvortuxizumab. Ecromeximab. Eculizumab, edobacomab, edrecolomab, efalizumab, efungumab, eldelumab, elezanumab, elgemtumab, elotuzumab, elsilimomab, emactuzumab, emapalumab, emibetuzumab, emicizumab, enapotamab, enavatuzumab, enfortumab, enlimomab, enoblituzumab, enokizumab, enoticumab. Ensituximab, epcoritamab, epitumomab, epratuzumab, eptinezumab, erenumab, erlizumab, ertumaxomab, etaracizumab, etesevimab, etigilimab, etrolizumab, evinacumab, evolocumab, exbivirumab, fanolesomab, faralimomab, faricimab, farletuzumab, fasinumab, felvizumab, fezakinumab, fibatuzumab, ficlatuzumab, figitumumab, firivumab, flanvotumab, fletikumab, flotetuzumab, fontolizumab, foralumab, foravirumab, fremanezumab, fresolimumab, frovocimab, frunevetmab, fulranumab, futuximab, galcanezumab, galiximab, gancotamab, ganitumab, gantenerumab, gatipotuzumab, gavilimomab, gedivumab, gemtuzumab, gevokizumab, gilvetmab, gimsilumab, girentuximab, glembatumumab, glofitamab, golimumab, gomiliximab, gosuranemab, guselkumab, ianalumab, ibalizumab, ibritumomab, icrucumab, ifabotuzumab, igovomab, iladatuzumab, imalumab, imaprelimab, imeiromab, imdevimab, imgatuzumab, inclacumab, indatuximab, indusatumab, inebilizumab, infliximab, intetumumab, inolimomab, inotuzumab, ipilimumab, iomab-B, iratumumab, isatuximab, iscalimab, istiratumab, itolizumab, ixekizumab, keliximab, labetuzumab, lacnotuzumab, ladiratuzumab, lampalizumab, lanadelumab, landogrozumab, laprituximab, larcaviximab, lebrikizumab, lecanemab, lemalesomab, lendalizumab, lenvervimab, lenzilumab, lerdelimumab, leronlimab, lesofavumab, letolizumab, lexatumumab, libivirumab, lifastuzumab, ligelizumab, loncastuximab, losatuxizumab, lilotomab, lintuzumab, lirilumab, lodelcizumab, lokivetmab, lorvotuzumab, lucatumumab, lulizumab, lumiliximab, lumretuzumab, lupartumab, luspatercept, lutikizumab, maftivimab, mapatumumab, margetuximab, marstacimab, maslimomab, mavrilimumab, matuzumab, mepolizumab, metelimumab, milatuzumab, minretumomab, mirikizumab, mirvetuximab, mitumomab, modotuximab, mogamulizumab, monalizumab, morolimumab, mosunetuzumab, motavizumab, moxetumomab, muromonab-CD3, nacolomab, namilumab, naptumomab, naratuximab, narnatumab, natalizumab, navicixizumab, navivumab, naxitamab, nebacumab, necitumumab, nemolizumab, nerelimomab, nesvacumab, netakimab, nimotuzumab, nirsevimab, nivolumab, nofetumomab, obiltoxaximab, obinutuzumab, ocaratuzumab, ocrelizumab, odesivimab, odulimomab, ofatumumab, olaratumab, oleclumab, olendalizumab, olokizumab, omalizumab, omburtamab, onartuzumab, ontuxizumab; onvatilimab, opicinumab, oportuzumab, oregovomab, orticumab, otelixizumab, otilimab, otlertuzumab, oxelumab, ozanezumab, ozoralizumab, pagibaximab, palivizumab, pamrevlumab, panitumumab, pankomab, panobacumab, parsatuzumab, pascolizumab, pasotuxizumab, pateclizumab, patritumab, pembrolizumab, pemtumomab, perakizumab, pertuzumab, pexelizumab, pidilizumab, pinatuzumab, pintumomab, placulumab, prezalumab, plozalizumab, pogalizumab, polatuzumab, ponezumab, porgaviximab, prasinezumab, prezalizumab, priliximab, pritoxaximab, pritumumab, quilizumab, racotumomab, radretumab, rafivirumab, ralpancizumab, ramucirumab, ranevetmab, ranibizumab, raxibacumab, ravagalimab, ravulizumab, refanezumab, regavirumab, regdanvimab, relatlimab, remtolumab, reslizumab, retifanlimab, rilotumumab, rinucumab, risankizumab, rituximab, rivabazumab, robatumumab, rmab, roledumab, romilkimab, romosozumab, rontalizumab, rosmantuzumab, rovalpituzumab, rovelizumab, rozanolixizumab, ruplizumab, sacituzumab, samalizumab, samrotamab, sarilumab, satralizumab, satumomab, secukinumab, selicrelumab, seribantumab, setoxaximab, setrusumab, sevirumab, sibrotuzumab, sifalimumab, siltuxima, simtuzumab, siplizumab, sirtratumab, sirukumab, sofituzumab, solanezumab, solitomab, sonepcizumab, sontuzumab, sotrovimab, spartalizumab, spesolimab, stamulumab, sulesomab, suptavumab, sutimlimab, suvizumab, suvratoxumab, tabalumab, tacatuzumab, tadocizumab, tafasitamab, talacotuzumab, talizumab, talquetamab, tamtuvetmab, tanezumab, taplitumomab, tarextumab, tavolimab, teclistamab, tefibazumab, telimomab, telisotuzumab, telisotuzumab, tenatumomab, teneliximab, teplizumab, tepoditamab, teprotumumab, tesidolumab, tetulomab, tezepelumab, TGN1412, tibulizumab, tildrakizumab, tigatuzumab, timigutuzumab, timolumab, tiragolumab, tiragotumab, tislelizumab, tisotumab, tixagevimab, TNX-650, tocilizumab, tomuzotuximab, toralizumab, tosatoxumab, tositumomab, tovetumab, tralokinumab, trastuzumab, TRBS07, tregalizumab, tremelimumab, trevogrumab, tucotuzumab, tuvirumab, ublituximab, ulocuplumab, urelumab, urtoxazumab, ustekinumab, utomilumab, vadastuximab, vanalimab, vandortuzumab, vantictumab, vanucizumab, vapaliximab, varisacumab, varlilumab, vatelizumab, vedolizumab, veltuzumab, vepalimomab, vesencumab, vilobelimab, visilizumab, vobarilizumab, volociximab, vonlerolizumab, vopratelimab, vorsetuzumab, votumumab, vunakizumab, xentuzumab, XMAB-5574, zalutumumab, zanolimumab, zatuximab, zenocutuzumab, ziralimumab, zolbetuximab, or zolimomab.

The construction of product-producing recombinant cells (e.g., hybridoma cells and CHO cells) is well known in the art. In some embodiments, a product can be a recombinant protein, e.g., a recombinant antibody, including, e.g., a multispecific antibody. In some embodiments, a product can be a secreted product, which is secreted by a cell into the cell culture medium. In some embodiments, a product can be an intracellular product, which, once produced by the cell, remains contained within the cell, or remains contain within a cell wall, as is the case with certain production microorganisms, such as, e.g., certain bacteria. In some embodiments, a product can be a cellular organelle, or even a cell itself, as in the case of a cell therapy product.

Methods in accordance with embodiments involve initiating an inoculation cell culture having a starting cell density that ranges from about 0.25 million (×10⁶) cells/mL up to about 25×10⁶ cells/mL, such as about 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24 or 24.5 million cells/mL. Methods in accordance with various embodiments involve initiating an inoculation cell culture having a starting cell density that ranges from about 20×10⁶ cells/mL up to about 25×10⁶ cells/mL. Methods in accordance with some embodiments involve initiating an inoculation cell culture having a starting density of about 22.5×10⁶ cells/mL. In some embodiments, the methods involve initiating an inoculation cell culture having a starting cell density that ranges from about 0.5 to about 2 million cells/mL. In some embodiments, the methods involve initiating an inoculation cell culture having starting cell density that ranges from about 2 to about 5 million cells/mL. In some embodiments, the methods involve initiating an inoculation cell culture having a starting cell density that ranges from about 5 to about 7.5 million cells/mL. In some embodiments, the methods involve initiating an inoculation cell culture having a starting cell density that ranges from about 7.5 to about 10 million cells/mL.

In certain embodiments, the method of cell culture provided herein maintains a viability percentage of at least 40%, 45%, 50%, 55%, 60%, 65%, 70%, 75%, 80%, or 85% throughout performance of the cell culturing process, e.g., 85%, 86%, 87%, 88%, 89%, 90%, 91%, 92%, 93%, 94%, 95%, 96%, 97% or 98%.

In some embodiments, the methods involve initiating an inoculation cell culture having a starting cell density that ranges from about 0.1% packed cell volume (PCV) up to about 4% PCV, such as about 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8 or 3.9% PCV. In some embodiments, an inoculation cell culture has an initial % PCV that ranges from 0.1 to 0.5% PCV. In some embodiments, an inoculation cell culture has an initial % PCV that ranges from 0.5 to 1%. In some embodiments, an inoculation cell culture has an initial % PCV that ranges from 1 to 1.5%. In some embodiments, an inoculation cell culture has an initial % PCV that ranges from 1.5 to 2%. In some embodiments, an inoculation cell culture has an initial % PCV that ranges from 2 to 2.5%. In some embodiments, an inoculation cell culture has an initial % PCV that ranges from 2.5 to 3%. In some embodiments, an inoculation cell culture has an initial % PCV that ranges from 3 to 3.5%. In some embodiments, an inoculation cell culture has an initial % PCV that ranges from 3.5 to 4%. In some embodiments, the methods involve initiating a new cell culture having an initial cell density that ranges from about 0.10% packed cell volume (PCV) up to about 10% PCV, such as about 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, or 9.8% PCV.

Methods in accordance with embodiments described herein involve growing an inoculation cell culture, as described above, to a first target cell density prior to conducting a perfusion procedure on at least a portion of the inoculation cell culture. In some embodiments, the first target cell density ranges from about 50×10⁶) cells/mL up to about 150×10⁶ cells/mL, such as about 60, 70, 80, 90, 100, 110, 120, 130 or 140×10⁶ cells/mL. In some embodiments, the methods involve growing an inoculation cell culture to a target cell density that ranges from about 50 to about 100 million cells/mL. In some embodiments, the methods involve growing an inoculation cell culture to a target cell density that ranges from about 60 to about 100 million cells/mL. In some embodiments, the methods involve growing an inoculation cell culture to a target cell density that ranges from about 100×10⁶ cells/mL to about 150×10⁶ cells/mL.

In some embodiments, the methods involve growing an inoculation cell culture, e.g., building cell mass or achieving a target cell density that ranges from about 10% packed cell volume (PCV) up to about 30% PCV, such as about 11, 12, 13, 14, 15, 16, 17, 18, 19, 20, 21, 22, 23, 24, 25, 26, 27, 28 or 29% PCV. In some embodiments, the methods involve growing an inoculation cell culture to a target cell density that ranges from about 10 to about 15% PCV. In some embodiments, the methods involve growing an inoculation cell culture to a target cell density that ranges from about 15 to about 20% PCV. In some embodiments, the methods involve growing an inoculation cell culture to a target cell density that ranges from about 20 to about 25% PCV. In some embodiments, the methods involve growing an inoculation cell culture to a target cell density that ranges from about 25 to about 30% PCV.

In certain embodiments, the cell culture (e.g., inoculation cell culture) produced by the methods described herein is part of a seed train in pre-production cell culturing. For example, a cell culture produced by the methods described herein may be used to seed another, larger, bioreactor that constitutes a further step in pre-production cell culture. In specific embodiments, the cells in said cell culture are concentrated using a centrifuge, e.g., a continuous flow centrifuge, and the resulting concentrated cells (that is, the solids phase or heavy phase) are used to initiate another cell culture vessel, e.g., bioreactor. In certain embodiments, the bioreactor is a pre-production bioreactor. In certain other embodiments, the bioreactor is a production bioreactor.

The cell culture methods described herein may further be used to initiate multiple downstream cell cultures so that cell mass may be built in parallel, or so that product production may be conducted in multiple vessels in parallel. In specific embodiments, for example, the solids phase or heavy phase is used to initiate more than one, e.g., 2, 3 or more other cell cultures in 2, 3 or more cell culture vessels, e.g., bioreactors. Such other cell cultures may be, e.g., additional (downstream) pre-production cell cultures, or may be production cell cultures. In certain specific embodiments, the heavy phase or solids phase is used to initiate 2 or 3 or more pre-production cell cultures. In certain specific embodiments, the heavy phase or solids phase is used to initiate 2 or 3 or more production cell cultures. Such initiation may comprise collecting the heavy phase or solids phase after centrifugation is complete, dividing it into the number of pre-production or production cultures to be initiated, and initiating said cultures. Such initiation may also comprise collecting the heavy phase or solids phase continuously or discontinuously during centrifugation, and during centrifugation, dividing it into the number of pre-production or production cultures to be initiated, and initiating said cultures.

Methods in accordance with embodiments described herein involve initiating a production cell culture having a starting cell density that ranges from about 0.25×10⁶ cells/mL up to about 25×10⁶ cells/mL, such as about 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24 or 24.5×10⁶ cells/mL. In some embodiments, the methods involve initiating a production cell culture having a starting cell density that ranges from about 0.5×10⁶ cells/mL to about 2×10⁶ cells/mL. In some embodiments, the methods involve initiating a production cell culture having a starting cell density that ranges from about 2×10⁶ cells/mL to about 5×10⁶ cells/mL. In some embodiments, the methods involve initiating a production cell culture having a starting cell density that ranges from about 5×10⁶ cells/mL to about 7.5×10⁶ cells/mL. In some embodiments, the methods involve initiating a production cell culture having a starting cell density that ranges from about 7.5×10⁶ cells/mL to about 10×10⁶ cells/mL. In some embodiments, the methods involve initiating a production cell culture having a starting cell density that ranges from about 10×10⁶ cells/mL to about 12.5×10⁶ cells/mL. In some embodiments, the methods involve initiating a production cell culture having a starting cell density that ranges from about 12.5×10⁶ cells/mL to about 15×10⁶ cells/mL. In some embodiments, the methods involve initiating a production cell culture having a starting cell density that ranges from about 15×10⁶ cells/mL to about 17.5×10⁶ cells/mL. In some embodiments, the methods involve initiating a production cell culture having a starting cell density that ranges from about 17.5×10⁶ cells/mL to about 20×10⁶ cells/mL. In some embodiments, the methods involve initiating a production cell culture having a starting cell density that ranges from about 20×10⁶ cells/mL to about 22.5×10⁶ cells/mL. In some embodiments, the methods involve initiating a production cell culture having a starting cell density that ranges from about 22.5×10⁶ cells/mL to about 25×10⁶ cells/mL.

Methods in accordance with embodiments described herein involve growing a production cell culture, as described above, to a first target cell density prior to conducting a perfusion procedure on at least a portion of the production cell culture. In some embodiments, the first target cell density ranges from about 50×10⁶ cells/mL up to about 150×10⁶ cells/mL, such as about 60, 70, 80, 90, 100, 110, 120, 130 or 140×10⁶ cells/mL. In some embodiments, the methods involve growing a production cell culture to a target cell density that ranges from about 50×10⁶ cells/mL to about 100×10⁶ cells/mL. In some embodiments, the methods involve growing a production cell culture to a target cell density that ranges from about 60×10⁶ cells/mL to about 100×10⁶ cells/mL. In some embodiments, the methods involve growing a production cell culture to a target cell density that ranges from about 100×10⁶ cells/mL to about 150×10⁶ cells/mL.

In some embodiments, the methods involve building cell mass or achieving a first target cell density that ranges from about 10% packed cell volume (PCV) up to about 30% PCV, such as about 11, 12, 13, 14, 15, 16, 17, 18, 19, 20, 21, 22, 23, 24, 25, 26, 27, 28 or 29% PCV. In some embodiments, the methods involve growing a production cell culture to a target cell density that ranges from about 10 to about 15% PCV. In some embodiments, the methods involve growing a production cell culture to a target cell density that ranges from about 15 to about 20% PCV. In some embodiments, the methods involve growing a production cell culture to a target cell density that ranges from about 20 to about 25% PCV. In some embodiments, the methods involve growing a production cell culture to a target cell density that ranges from about 25 to about 30% PCV.

Aspects of the technologies described herein involve conducting a perfusion procedure on a least a portion of a cell culture (e.g., an inoculation cell culture or a production cell culture) using a continuous flow centrifuge. Continuous flow centrifuges are known in the art, and are described, for example, in PCT Publication No. WO2008/138345 and PCT Publication No. WO2020/229184, the disclosures of which are incorporated by reference herein in their entireties. Continuous flow centrifuges that can be used in accordance with embodiments described herein, but are not limited to, disc stack centrifuges, tubular centrifuges, and the like. In certain embodiments, a continuous flow centrifuge is configured for sterilization, meaning that one or more components of the centrifuge (e.g., the bowl) can be subjected to a sterilization procedure, making it suitable for use under biomanufacturing conditions (e.g., in accordance with cGMP regulations). In certain embodiments, a continuous flow centrifuge comprises a disposable component, such as a disposable bowl component. In such embodiments, the disposable component is configured to be easily separated from the remaining components of the device, and can be readily discarded and/or replaced. A new disposable component can then be attached to the centrifuge. The new disposable component can be presterilized, or can be configured for sterilization, either separately from the remaining components of the centrifuge, or together with the remaining components of the centrifuge.

Continuous flow centrifuges in accordance with embodiments of the described herein are generally operated and flow rates suitable for cell separation in the solids phase from cell medium in the liquid phase. Flow rate, in addition to the impact it has on separation, has an impact on the residence time of cells outside of the production bioreactor. Increasing flow rate can minimize residence time while optimally still enabling the intended separation of cells.

Continuous flow centrifuges in accordance with embodiments of the described herein also generally include suitable intake and outlet components to allow for their operable connection to one or more bioreactors, one or more cell culture medium reservoirs, etc. Further, continuous flow centrifuges in accordance with embodiments described herein may be generally configured for interoperability with standard cell culture manufacturing equipment, such as pumps, controllers, flow meters, and the like. Continuous flow centrifuges described herein are generally operated at flow rates suitable for cell separation, where separation is achieved between a solids phase (cells) and a liquid phase (cell medium). In certain embodiments, suitable intake and outlet components are designed to minimize the volume of the system and thus residence time of cells outside of the cell culture vessel bioreactor, e.g., production bioreactor. In certain embodiments, the volume of the centrifugation system (including inlet and outlet components) may be accomplished by minimizing the length and/or diameter of piping, tubing, or tubing flow kits, while still enabling the intended flow rates of the system.

Continuous flow centrifuges in accordance with embodiments of the described herein may also generally include elements of temperature control to provide suitable conditions for the material exiting the production bioreactor and entering the centrifugation system, during centrifugation within the system, the resulting solids phase exiting the system before returning to the production bioreactor, and the resulting liquid phase exiting the system before being processed further downstream. Temperature control elements may be passive or actively controlled. In one embodiment, the temperature control is passive. Here, the piping, tubing, or tubing flow kits may be insulated or non-insulated (as desired) allowing for passive temperature control to an ambient temperature, or to a decreased temperature present at the point of operation. For e.g., chilled water (at about 4° C. to 10° C.) may be used to cool the water which in turn cools centrifuge seals and minimizes the temperature gains from centrifuge spinning. In a specific embodiment, the ambient temperature is reduced to minimize temperature gains from frictional forces at high bowl speeds for cell separation. Piping, tubing, or tubing flow kits may be insulated or non-insulated allowing for passive temperature control via the ambient temperature at the point of operation.

In other embodiments, the temperature control is active. For example, piping, tubing, or tubing flow kits may be subjected to heat exchangers allowing for active temperature control to specific temperature setpoints. The centrifuge may have temperature control for the centrifuge bowl to minimize temperature gain from frictional forces at high bowl speeds necessary for cell separation. The centrifuge may have temperature control for the mechanical seals to minimize temperature gain from frictional forces at high bowl speeds necessary for cell separation. Further, the temperature control for the centrifuge bowl and mechanical seals may be provided as ambient temperature or decreased temperature to minimize temperature increases during processing.

In certain embodiments of any of the methods disclosed herein, residence time of the cells and cell culture medium is minimized such that the viability of the cells in the cell culture is maintained, or is not substantially reduced. In certain embodiments, the residence time is less than 3 minutes, 2 minutes, or 1 minute. In certain embodiments, residence time is reduced by increasing the flow rate of culture medium and cells from culture vessel to centrifuge and back to culture vessel. In other embodiments, residence time is reduced through use of larger conduiting, e.g. tubing, between culture vessel and centrifuge, use of a larger centrifuge, and the like. In certain embodiments, a particular residence time results in no more than a 3%, 2%, 1% or 0.5% loss of cell viability during culturing.

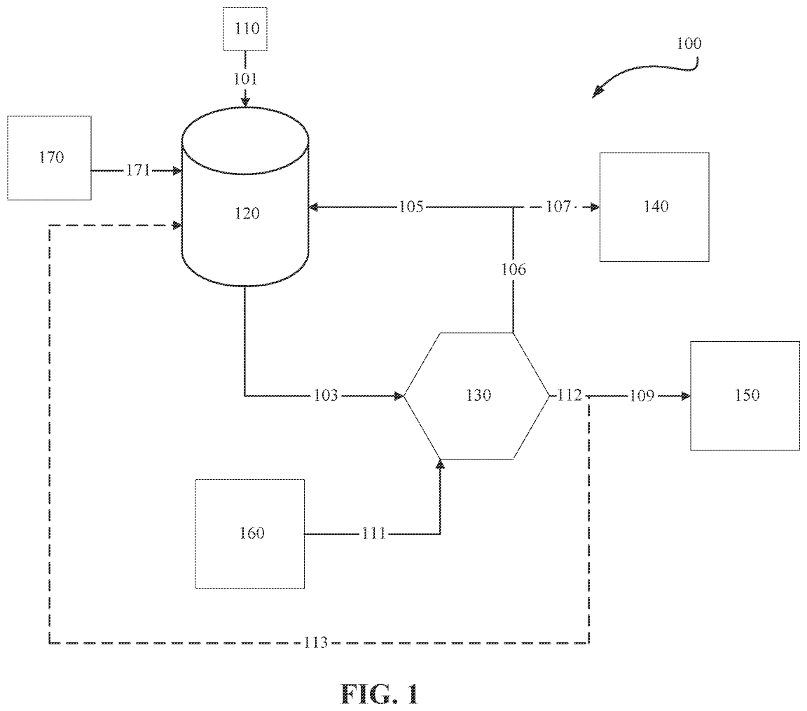

In certain embodiments of any of the methods disclosed herein, the temperature of the cell culture medium is adjusted or maintained during residence time. In specific embodiments, during residence time temperature is maintained such that any temperature transient is 4° C. or less, 3° C. or less, 2° C. or less, or 1° C. or less during residence time, as compared to the temperature of medium in the cell culture vessel. In various embodiments, the temperature of the cell culture medium is maintained during residence time using a water bath or heat exchanger, e.g., during cell culture medium transit between culture vessel and centrifuge, between centrifuge and cell culture vessel, or both. In FIG. 1, for example, the temperature of cell culture medium may be maintained, such that any temperature transient is 4° C. or less, 3° C. or less, 2° C. or less, or 1° C. or less during transit from cell culture vessel 120, through sterile connection 103, cell separation device 130, solids phase outlet 106, and through sterile connection 105 back to cell culture vessel 120. In specific embodiments, the cell culture medium is maintained during residence time at a temperature of between about 30° C. and about 39° C., e.g., between 31° C. and 38° C., between 32° C. and 38° C., between 33° C. and 38° C., between 34° C. and 38° C., between 35° C. and 38° C. or between 36° C. and 38° C. In certain embodiments, the centrifuge, e.g., continuous centrifuge, e.g., single-use centrifuge, comprises a water cooling system that reduces heat addition to the cell culture medium within the centrifuge, e.g., heat addition at the seals.

In some embodiments, a continuous flow centrifuge can be characterized by a sigma factor, or sigma value, which refers to an operation constant that represents the geometry and speed of the centrifuge. A sigma factor can be used to compare two or more centrifuges having different sizes, geometries, and/or operating speeds, such that two different centrifuges having different operating parameters can be compared to each other in order to achieve similar operational performance. Continuous flow centrifuges in accordance with embodiments of the technologies generally have a sigma factor that ranges from about 1,000 m² to about 200,000 m², such as about 1,500, 2,000, 2,500, 3,000, 3,500, 4,000, 4,500, 5,000, 5,500, 6,000, 6,500, 7,000, 7,500, 8,000, 8,500, 9,000, 9,500, 10,000, 12,000, 14,000, 16,000, 18,000, 20,000, 25,000, 30,000, 35,000, 40,000, 45,000, 50,000, 55,000, 60,000, 65,000, 70,000, 75,000, 80,000, 85,000, 90,000, 95,000, 100,000, 120,000, 140,000, 160,000, or 180,000 m². Continuous flow centrifuges in accordance with embodiments of the technologies described herein are generally configured to achieve an operating speed that ranges from about 3,000 to about 10,000 RPM, such as about 3,500, 4,000, 4,500, 5,000, 5,500, 6,000, 6,500, 7,000, 7,500, 8,000, 8,500, 9,000 or 9,500 RPM. Some single-use continuous flow centrifuges may operate at speeds ranging from about 3,000 RPM to about 6,000 RPM.

In use, a continuous flow centrifuge in accordance with embodiments of the technologies described herein may be configured to receive at least a portion of a cell culture (e.g., an inoculation cell culture or a production cell culture) and to separate the received portion of the cell culture into a solids phase, containing all or a substantial portion of the cells in the received portion, and a liquid phase, containing mostly cell culture medium. In certain embodiments, the solids phase can have a final cell density that ranges from about 1% PCV to about 10%, about 20%, about 30%, about 40% or about 50% PCV.

Following separation into the solids and liquid phases, the centrifuge and/or ancillary components thereof are configured to return the solids phase, or a portion thereof, as well as, optionally, a portion of the liquid phase, to a new bioreactor (or to the original bioreactor) in order to sustain, maintain and/or initiate a new cell culture. In addition to the solids phase, and optionally, a portion of the liquid phase, a suitable quantity of fresh cell culture medium can be combined with the solids phase (and optionally a portion of the liquid phase) in order to achieve a desired initial cell density for the new cell culture. In some embodiments, the initial cell density ranges from about 0.25×10⁶ cells/mL up to about 25×10⁶ cells/mL, such as about 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24 or 24.5×10⁶ cells/mL. In some embodiments, the methods involve initiating a new cell culture having an initial cell density that ranges from about 0.5×10⁶ cells/mL to about 2×10⁶ cells/mL. In some embodiments, the methods involve initiating a new cell culture having an initial cell density that ranges from about 2×10⁶ cells/mL to about 5×10⁶ cells/mL. In some embodiments, the methods involve initiating a new cell culture having an initial cell density that ranges from about 5×10⁶ cells/mL to about 7.5×10⁶ cells/mL. In some embodiments, the methods involve initiating a new cell culture having an initial cell density that ranges from about 7.5×10⁶ cells/mL to about 10×10⁶ cells/mL. In some embodiments, the methods involve initiating a production cell culture having an initial cell density that ranges from about 10×10⁶ cells/mL to about 12.5×10⁶ cells/mL. In some embodiments, the methods involve initiating a production cell culture having an initial cell density that ranges from about 12.5×10⁶ cells/mL to about 15×10⁶ cells/mL. In some embodiments, the methods involve initiating a production cell culture having an initial cell density that ranges from about 15 to about 17.5×10⁶ cells/mL. In some embodiments, the methods involve initiating a production cell culture having an initial cell density that ranges from about 17.5×10⁶ cells/mL to about 20×10⁶ cells/mL. In some embodiments, the methods involve initiating a production cell culture having an initial cell density that ranges from about 20×10⁶ cells/mL to about 22.5×10⁶ cells/mL. In some embodiments, the methods involve initiating a production cell culture having an initial cell density that ranges from about 22.5×10⁶ cells/mL to about 25×10⁶ cells/mL.

In some embodiments, the methods involve initiating a new cell culture having an initial cell density that ranges from about 0.1% packed cell volume (PCV) up to about 10% PCV, such as about 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, or 9.8% PCV. In some embodiments, a new cell culture has an initial % PCV that ranges from 0.1 to 1%. In some embodiments, a new cell culture has an initial % PCV that ranges from 1.1 to 2%. In some embodiments, a new cell culture has an initial % PCV that ranges from 2.1 to 3%. In some embodiments, a new cell culture has an initial % PCV that ranges from 3.1 to 4%. In some embodiments, a new cell culture has an initial % PCV that ranges from 4.1 to 5%. In some embodiments, a new cell culture has an initial % PCV that ranges from 5.1 to 6%. In some embodiments, a new cell culture has an initial % PCV that ranges from 6.1 to 7%. In some embodiments, a new cell culture has an initial % PCV that ranges from 7.1 to 8%. In some embodiments, a new cell culture has an initial % PCV that ranges from 8.1 to 9%. In some embodiments, a new cell culture has an initial % PCV that ranges from 9.1 to 10%.

Methods in accordance with embodiments of the technologies described herein may involve growing a new cell culture to a target cell density prior to conducting a perfusion procedure on at least a portion of the cell culture. In some embodiments, the target cell density ranges from about 50 million (×10⁶) cells/mL up to about 150 million cells/mL, such as about 60, 70, 80, 90, 100, 110, 120, 130 or 140 million cells/mL. In some embodiments, the methods involve growing a new cell culture to a target cell density that ranges from about 50 to about 100 million cells/mL. In some embodiments, the methods involve growing a new cell culture to a target cell density that ranges from about 60 to about 100 million cells/mL. In some embodiments, the methods involve growing a new cell culture to a target cell density that ranges from about 100 to about 150 million cells/mL.

In some embodiments, the methods involve growing a new cell culture, building cell mass or achieving a target cell density that ranges from about 10% packed cell volume (PCV) up to about 30% PCV, such as about 11, 12, 13, 14, 15, 16, 17, 18, 19, 20, 21, 22, 23, 24, 25, 26, 27, 28 or 29% PCV. In some embodiments, the methods involve growing a new cell culture to a target cell density that ranges from about 10 to about 15% PCV. In some embodiments, the methods involve growing a new cell culture to a target cell density that ranges from about 15 to about 20% PCV. In some embodiments, the methods involve growing a new cell culture to a target cell density that ranges from about 20 to about 25% PCV. In some embodiments, the methods involve growing a new cell culture to a target cell density that ranges from about 25 to about 30% PCV.

Various methods described herein may be used to achieve a desired rate of perfusion for the cell culture. The rate of perfusion can be any rate appropriate to the cell culture. For example, the rate of perfusion can range from about 0.5 vessel volumes per day (VVD) to about 10 VVD, such as about 0.6, 0.7, 0.8, 0.9, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5 or about 9.75 VVD. In some embodiments, the rate of perfusion ranges from about 0.5 to about 6 VVD, e.g., 0.5 to about 4 VVD, or from about 0.7 VVD to about 6 VVD. In some embodiments, the rate of perfusion ranges from about 4 to about 6 VVD. In some embodiments, the rate of perfusion ranges from about 6 to about 8 VVD. In some embodiments, the rate of perfusion ranges from about 8 to about 10 VVD.

Aspects of the disclosure include variations on the perfusion procedure, which can be used to achieve any of a variety of perfusion rates for the cell culture. For example, the rate of perfusion can remain constant over a period of time, or can be altered (i.e., increased or decreased) over the course of a period of perfusion, or any combination thereof. Further, an increase or decrease in the rate of perfusion can be applied in any manner known in the art, including, but not limited to, a steady alteration over time, e.g., a steady increase during a period of perfusion, or a series of alterations over time, e.g., a series of steady alterations, a series of stepwise alterations (e.g., the rate of perfusion could be increased or decreased in a stepwise manner), or any combination thereof. The perfusion can be applied in a continuous manner or in an intermittent manner, as noted above. The timing of the initiation and cessation of a perfusion period(s), and of any alterations to the perfusion, can be predetermined, e.g., at a set time(s) or interval(s), or based upon the monitoring of some parameter or criterion of the cell culture.

Perfusion procedures in accordance with embodiments of the disclosure can be conducted over a time period that can range from several hours to several days. For example, in some embodiments, a time period over which a perfusion procedure, or a cell culture method comprising such perfusion procedure, is conducted ranges from 0.5 hours up to about 5 hours, such as about 1, 1.5, 2, 2.5, 3, 3.5, 4 or 4.5 hours or more. In some embodiments, a time period over which a perfusion procedure, or a cell culture method comprising such perfusion procedure, is conducted ranges from about 5 hours up to about 24 hours, such as about 6, 7, 8, 9, 10, 11, 12, 13, 14, 15, 16, 17, 18, 19, 20, 21, 22, or 23 hours. In some embodiments, a time period over which a perfusion procedure, or a cell culture method comprising such perfusion procedure, is conducted ranges from about 1 day up to about 20 days, such as about 2, 3, 4, 5, 6, 7, 8, 9, 10, 11, 12, 13, 14, 15, 16, 17, 18 or 19 days. In other embodiments, a perfusion procedure, or the cell culture method comprising the perfusion procedure, is conducted up to 20, 25, 30, 35, 40, 45, 50, 55, 60, 65, 70, 75, 80, 85 or 90 days. Moreover, perfusion procedures in accordance with the above embodiments may be conducted in a semi-continuous, discontinuous or “punctuated” manner, wherein a perfusion procedure is conducted, for example, once per day over a period of several days.

Methods and systems in accordance with embodiments described herein may be designed to maintain a target cell viability throughout a perfusion procedure, so that the cell culture is able to maintain a cell viability that is at or above a desired target value. In some embodiments, the methods result in a cell culture viability that is greater than or equal to 40%, 45%, 50%, 55%, 60%, 65%, 70%, 75%, 80%, or 85%, e.g., 85%, 86%, 87%, 88%, 89%, 90%, 91%, 92%, 93%, 94%, 95%, 96%, 97% or 98% throughout the duration of a perfusion procedure.

Methods and systems in accordance with embodiments of the technologies described herein may be designed to maintain a target cell viability throughout the duration of a cell culture, so that the cell culture is able to maintain a cell viability that is at or above a desired target value for the duration of the entire culture. In some embodiments, the methods result in a cell culture viability that is greater than or equal to 40%, 45%, 50%, 55%, 60%, 65%, 70%, 75%, 80%, 85%, 90%, 95%, or 99% throughout the duration of the entire cell culture.

Methods and systems in accordance with embodiments of the technologies described herein may be designed to maintain a target concentration of a cell culture waste product, or byproduct, at or below a target level. For example, in some embodiments, the methods result in a cell culture lactic acid (lactate) concentration that is less than or equal to 4 g/L, such as less than or equal to 3.75, 3.5, 3.25, 3.0, 2.75, 2.5, 2.25, 2.0, 1.75, 1.5, 1.25, 1.0, 0.75, 0.5, 0.25, 0.2, or 0.1 g/L throughout the duration of a perfusion procedure. In some embodiments, the methods result in a cell culture ammonium concentration that is less than or equal to 4 mM, such as less than or equal to 3.75, 3.5, 3.25, 3.0, 2.75, 2.5, 2.25, 2.0, 1.75, 1.5, 1.25, 1.0, 0.75, 0.5, 0.25, 0.2, or 0.1 mM throughout the duration of a perfusion procedure. In some embodiments, the methods result in a cell culture pCO₂ concentration that is less than or equal to 150 mmHg, such as less than or equal to 140, 130, 120, 110, 100, 90, 80, 70, 60, 50, 40, 30, 20 or 10 mmHg throughout the duration of a perfusion procedure.

In some embodiments, the methods and systems described herein are configured for industrial scale use at cell culture bioreactor volumes that are, or are greater than, about 20 L, such as 50, 80, 100, 250, 300, 350, 400, 450, 500, 1,000, 2,000, 2,500, 3,000, 3,500, 4,000, 4,500, 5,000, 7,500, 8,000, 10,000, 12,000, 15,000, 16,000, 18,000, 20,000, 22,000, 24,000, 26,000, 28,000 or 30,000 liters or more. In some embodiments, a cell culture bioreactor has a working volume that is greater than or equal to 80 L. In some embodiments, a cell culture bioreactor has a total volume that ranges from 100 L to 3,000 L. In some embodiments, a cell culture bioreactor has a total volume of 100 L. In some embodiments, a cell culture bioreactor has a total volume of 3,000 L. In some embodiments, a cell culture bioreactor has a total volume that ranges from 100 L to 30,000 L. In some embodiments, a cell culture bioreactor has a total volume that ranges from 5,000 L to 30,000 L. In some embodiments a cell culture bioreactor has a total volume that ranges from 100 L to 6,000 L.

Aspects of the described technologies may include methods that involve transferring at least a portion of a first cell culture from one bioreactor into another bioreactor to initiate a second cell culture. In certain embodiments, the methods involve combining a desired volume of the solids phase, which is discharged from the continuous flow centrifuge, with a desired volume of fresh cell culture medium, to sustain, maintain and/or initiate a new cell culture. In certain embodiments, the new cell culture is an inoculation cell culture. In certain embodiments, the new cell culture is a production cell culture.

In some embodiments, where the new cell culture is an inoculation cell culture, the methods involve further culturing the new inoculation cell culture for a period of time to generate a target cell density. In some embodiment, the new inoculation cell culture is cultured for a period of time that ranges from 1 to 7 days, such as 2, 3, 4, 5 or 6 days.

In some embodiments, where the new cell culture is a production cell culture, the methods further involve culturing the production cell culture under batch or fed-batch process conditions. In some embodiments, the methods further involve performing a harvesting procedure on the cell culture to separate the cells from the cell culture medium at a given time point, and conducting a purification procedure on the harvested cell culture fluid (HCCF). In some embodiments, the methods involve culturing a production culture for a period of time that ranges from 1 to 20 days, such as 2, 3, 4, 5, 6, 7, 8, 9, 10, 11, 12, 13, 14, 15, 16, 17, 18 or 19 days before conducting the harvesting procedure.

In one preferred embodiment, a method for culturing mammalian cells comprises placing a plurality of mammalian cells and a volume of cell culture medium in a culture vessel to generate a cell culture; culturing the cell culture to a cell density of greater than or equal to 1% packed cell volume (PCV); conducting a perfusion procedure on the cell culture, wherein the perfusion procedure comprises: transferring at least a portion of the cell culture to a continuous flow centrifuge; operating the continuous flow centrifuge to generate a solids phase having a final cell density of greater than or equal to 1% PCV, e.g., to about 10%, about 20%, about 30%, about 40% or about 50% PCV; and returning the solids phase and a volume of cell culture medium to the culture vessel to achieve a perfusion rate that ranges from 0.5 to 6 vessel volumes per day (VVD), such as at least 1, 2, 3, 4, or 5 VVD, wherein following completion of the perfusion procedure, the cell culture has a cell density of greater than or equal to 0.2% PCV. In some embodiments, the perfusion rate ranges from 2 to 4 VVD. In some embodiments, the perfusion rate can range from about 0.5 vessel volumes per day (VVD) to about 10 VVD, such as at least, or about, 0.6, 0.7, 0.8, 0.9, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75 VVD, or about 10.0 VVD.

In one preferred embodiment, a method comprises generating a culture of mammalian cells having a cell density of greater than or equal to 1% PCV, the method comprising: placing a plurality of mammalian cells and a volume of culture medium in a culture vessel to generate a cell culture; culturing the cell culture to a cell density of that ranges from 10% to 30% PCV, such as 10% to 15% PCV, 15% to 20% PCV, 20% to 25% PCV, or 25% to 30% PCV; conducting a perfusion procedure on the cell culture, wherein the perfusion procedure comprises: transferring at least a portion of the cell culture to a continuous flow centrifuge; operating the continuous flow centrifuge to generate a solids phase having a cell density greater than the cell culture % PCV, e.g., at least 1%, 2%, 3%, 4%, 5%, 6%, 7%, 8%, 9% or 10% greater than that of the cell culture, e.g., greater than or equal to 15% to 20% PCV, 20% to 25% PCV, or 25% to 30% PCV, 30% to 35% PCV, 35% to 40% PCV, or greater than 40% PCV; and returning at least a portion of the solids phase and a volume of cell culture medium to the culture vessel to achieve a perfusion rate that ranges from 0.7 to 6 vessel volumes per day (VVD), such as 1, 2, 3, 4, or 5 VVD, wherein following completion of the perfusion procedure, the cell culture has a cell density of greater than or equal to 0.2% PCV, e.g., 0.2% to about 30% PCV. Preferably the perfusion rate is from 3.5 to 6 VVD. More preferably, the perfusion rate is from 5 to 6 VVD.

In one embodiment, a method comprises generating an culture of mammalian cells comprising at least, or about, 4.8×10¹² cells, the method comprising: placing a plurality of mammalian cells and a volume of culture medium in a culture vessel having a working volume of 80 L to generate a cell culture having a starting cell density of greater than or equal to 1 million cells/mL; culturing the cell culture to a cell density of greater than or equal to 10% PCV; conducting a perfusion procedure on the cell culture, wherein the perfusion procedure comprises: transferring at least a portion of the cell culture to a continuous flow centrifuge having a sigma factor that ranges from 10,000 to 200,000 m², operating the continuous flow centrifuge to generate a solids phase having a cell density that ranges from about 1% to about 10%, about 20%, about 30%, about 40% or about 50% PCV; and returning at least a portion of the solids phase and a volume of cell culture medium to the culture vessel to achieve a perfusion rate that ranges from 0.5 to 6 VVD, wherein following completion of the perfusion procedure, the cell culture has a cell density of greater than or equal to 60×10⁶ cells/mL. In specific embodiments, the solids phase cell density ranges from about 20% to about 40%. In more specific embodiments, the cell density ranges from about 30% to about 35%.

In another embodiment, provided herein is a method comprising generating a culture of mammalian cells comprising at least 1.80×10¹⁴ cells, the method comprising: placing a plurality of mammalian cells and a volume of culture medium in a culture vessel having a working volume of 3000 L to generate a cell culture having a starting cell density of greater than or equal to 1×10⁶ cells/mL; culturing the cell culture to a cell density of greater than or equal to 10% PCV; conducting a perfusion procedure on the cell culture, wherein the perfusion procedure comprises: transferring at least a portion of the cell culture to a continuous flow centrifuge having a sigma factor that ranges from 10,000 to 200,000 m², operating the continuous flow centrifuge to generate a solids phase having a cell density that ranges from about 1% to about 10%, about 20%, about 30%, about 40% or about 50% PCV; and returning at least a portion of the solids phase and a volume of cell culture medium to the culture vessel to achieve a perfusion rate that ranges from 0.5 to 6 VVD, wherein following completion of the perfusion procedure, the cell culture has a cell density of greater than or equal to 60×10⁶ cells/mL. In specific embodiments, the solids phase cell density ranges from about 20% to about 40%. In more specific embodiments, the cell density ranges from about 30% to about 35%.

In one preferred embodiment, a method comprises generating a culture of mammalian cells comprising at least 2.16×10¹⁴ cells, the method comprising: placing a plurality of mammalian cells and a volume of culture medium in a culture vessel having a working volume of 3600 L to generate a cell culture having a starting cell density of greater than or equal to 1 million cells/mL; culturing the cell culture to a cell density of greater than or equal to 10% PCV; conducting a perfusion procedure on the cell culture, wherein the perfusion procedure comprises: transferring at least a portion of the cell culture to a continuous flow centrifuge having a sigma factor that ranges from 10,000 to 200,000 m², operating the continuous flow centrifuge to generate a solids phase having a cell density that ranges from about 1% to about 10%, about 20%, about 30%, about 40% or about 50% PCV; and returning at least a portion of the solids phase and a volume of cell culture medium to the culture vessel to achieve a perfusion rate that ranges from 0.5 to 6 VVD, wherein following completion of the perfusion procedure, the cell culture has a cell density of greater than or equal to 60 million cells/mL. In specific embodiments, the solids phase cell density ranges from about 20% to about 40%. In more specific embodiments, the cell density ranges from about 30% to about 35%.

Thus, provided herein are example embodiments of a method for culturing mammalian cells:

Embodiment 1: A method for culturing mammalian cells, comprising: (a) placing a plurality of mammalian cells and a volume of culture medium in a culture vessel to generate a cell culture; (b) culturing the cell culture to a cell density of greater than or equal to 1% packed cell volume (PCV); (c) during step (b), conducting a perfusion procedure on the cell culture, wherein the perfusion procedure comprises: (i) transferring at least a portion of the cell culture to a continuous flow centrifuge; (ii) operating the continuous flow centrifuge to generate a solids phase having a cell density of greater than or equal to 1% PCV, e.g. to about 10%, about 20%, about 30%, about 40% or about 50% PCV; and (iii) returning the solids phase and a volume of cell culture medium to the culture vessel to achieve a perfusion rate that ranges from 0.5 to 6 vessel volumes per day (VVD), wherein following completion of the perfusion procedure (e.g., after 7-8 days), the cell culture has a cell density of 0.2% PCV or greater, e.g., 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, or 9.8% PCV, or 10%, 15%, 20%, 25%, or 30% PCV. Embodiment 2: The method of Embodiment 1, wherein the perfusion rate ranges from 2 to 4 VVD. Embodiment 3: The method of Embodiment 1 or 2, wherein the perfusion procedure comprises increasing or decreasing the perfusion rate in a constant manner. Embodiment 4: The method of Embodiment 1 or 2, wherein the perfusion procedure comprises increasing or decreasing the perfusion rate in a variable manner. Embodiment 5: The method of any one of Embodiments 1-4, wherein the perfusion procedure is conducted continuously over a time period that ranges from 1 to 7 days. Embodiment 6: The method of any one of Embodiments 1-4, wherein the perfusion procedure is conducted in a semi-continuous manner over a time period that ranges from 1 to 7 days. Embodiment 7: The method of any one of Embodiments 1-6, wherein the continuous flow centrifuge comprises a disc stack bowl. Embodiment 8: The method of any one of Embodiments 1-6, wherein the continuous flow centrifuge comprises a tubular bowl. Embodiment 9: The method of any one of Embodiments 1-8, wherein the continuous flow centrifuge has an operating speed that ranges from 3,000 to 10,000 RPM. Embodiment 10: The method of any one of Embodiments 1-9, wherein the continuous flow centrifuge comprises a sterilizable component. Embodiment 11: The method of any one of Embodiments 1-10, wherein the continuous flow centrifuge comprises a disposable component. Embodiment 12: The method of any one of Embodiments 1-11, wherein the continuous flow centrifuge has a sigma value that ranges from 1,000 m² to 200,000 m². Embodiment 13: The method of any one of Embodiments 1-12, wherein following completion of the centrifugation procedure, the cell culture has a viability percentage that is greater than or equal to 80%, e.g., 80%, 81%, 82%, 83%, 84%, 85%, 86%, 87%, 88%, 89%, 90%, 91%, 92%, 93%, 94%, 95%, 96%, 97%, 98% or 99%. Embodiment 14: The method of any one of Embodiments 5-13, wherein the cell culture maintains a viability percentage that is greater than or equal to 85% over the time period of 1 to 7 days. Embodiment 15: The method of any of Embodiments 1-14, wherein the cell culture medium is maintained during residence time at a temperature of between about 30° C. and about 39° C., e.g., between 31° C. and 38° C., between 32° C. and 38° C., between 33° C. and 38° C., between 34° C. and 38° C., between 35° C. and 38° C. or between 36° C. and 38° C. Embodiment 16: The method of any one of Embodiment 1-15, wherein the mammalian cells comprise recombinant mammalian cells. Embodiment 17: The method of Embodiment 16, wherein the recombinant mammalian cells comprise recombinant Chinese hamster ovary (CHO) cells. Embodiment 18: The method of Embodiment 17, wherein following completion of the centrifugation procedure, the cell culture has a lactate concentration that is less than or equal to 4 g/L. Embodiment 19: The method of any one of claims 16-18, wherein the recombinant mammalian cells produce a secreted product. Embodiment 20: The method of Embodiment 16, wherein the secreted product comprises a recombinant protein. Embodiment 22: The method of Embodiment 21, wherein the recombinant protein is an antibody. Embodiment 23: The method of any one of Embodiments 1-22, wherein the culture vessel has a working volume that is greater than or equal to 80 L. Embodiment 24: The method of any one of embodiments 1-23, wherein the culture vessel has a total volume that ranges from 100 L to 30,000 L. Embodiment 25: The method of any one of Embodiments 1-23, wherein the culture vessel has a total volume that ranges from 5,000 L to 30,000 L. Embodiment 26: The method of any one of Embodiments 1-23, wherein the culture vessel has a total volume that ranges from 100 L to 6,000 L. Embodiment 27: The method of any one of Embodiments 1-26, further comprising transferring at least a portion of the cell culture to a different culture vessel to initiate a second cell culture. Embodiment 28: The method of Embodiment 27, wherein the second cell culture has an initial cell density that ranges from 0.1% to 10% PCV. Embodiment 29: The method of any one of Embodiments 1-28, further comprising transferring at least a portion of the cell culture to a production culture vessel to initiate a production culture having a starting cell density that ranges from 0.1% to 10% PCV. Embodiment 30: The method of Embodiment 29, further comprising culturing the production culture under batch or fed-batch process conditions. Embodiment 31: The method of any one of Embodiments 1-30, further comprising adding fresh culture fluid to the culture vessel.

Embodiment 33: A method of generating a culture of mammalian cells having a cell density of greater than or equal to 0.1% PCV, the method comprising: (a) placing a plurality of mammalian cells and a volume of cell culture medium in a culture vessel to generate a cell culture; (b) culturing the cell culture to a cell density of greater than or equal to 10% PCV; (c) conducting a perfusion procedure on the cell culture during step (b), wherein the perfusion procedure comprises: (i) transferring at least a portion of the cell culture to a continuous flow centrifuge; (ii) operating the continuous flow centrifuge to generate a solids phase having a cell density that ranges from about 1% to about 10%, about 20%, about 30%, about 40% or about 50% PCV; and (iii) returning a portion of the solids phase and a volume of cell culture medium to the culture vessel to achieve a perfusion rate that ranges from 0.5 to 6 VVD, wherein following completion of the perfusion procedure, the cell culture has a cell density of greater than or equal to 0.1% PCV. In certain embodiments, following completion of the perfusion procedure, the cell culture increases, or has increased, in cell density by greater than or equal to 0.1% PCV, e.g., greater than or equal to 0.1% to about 30% PCV. In specific embodiments, the solids phase cell density ranges from about 20% to about 40%. In more specific embodiments, the cell density ranges from about 30% to about 35%.

Embodiment 34: A method of generating a culture of mammalian cells comprising at least 4.8×10¹² cells, the method comprising: (a) placing a plurality of mammalian cells and a volume of cell culture medium in a culture vessel having a working volume of 80 L to generate a cell culture having a starting cell density of greater than or equal to 1×10⁶ cells/mL; (b) culturing the cell culture to a cell density of greater than or equal to 10% PCV; (c) conducting a perfusion procedure on the cell culture during step (b), wherein the perfusion procedure comprises: (i) transferring at least a portion of the cell culture to a continuous flow centrifuge comprising a disposable disc stack bowl and comprising a sigma factor that ranges from 1,000 to 200,000 m²; (ii) operating the continuous flow centrifuge to generate a solids phase having a cell density of greater than or equal to 1% PCV; and (iii) returning at least a portion of the solids phase and a volume of cell culture medium to the culture vessel to achieve a perfusion rate that ranges from 0.5 to 6 VVD, wherein following completion of the perfusion procedure, the cell culture has a cell density of greater than or equal to 60×10⁶ cells/mL.

Embodiment 35: A method of generating a culture of mammalian cells comprising at least 2.16×10¹⁴ cells, the method comprising: (a) placing a plurality of mammalian cells and a volume of cell culture medium in a culture vessel having a working volume of 3,000 L to generate a cell culture having a starting cell density of greater than or equal to 1 10⁶ cells/mL; (b) culturing the cell culture to a cell density of greater than or equal to 10% PCV; (c) conducting a perfusion procedure on the cell culture, wherein the perfusion procedure comprises: (i) transferring at least a portion of the cell culture to a continuous flow centrifuge comprising a disposable disc stack bowl and comprising a sigma factor that ranges from 1,000 to 200,000 m²; (ii) operating the continuous flow centrifuge to generate a solids phase having a cell density of greater than or equal to 1% PCV; and (iii) returning at least a portion of the solids phase and a volume of cell culture medium to the culture vessel to achieve a perfusion rate that ranges from 0.5 to 6 VVD, wherein following completion of the perfusion procedure, the cell culture has a cell density of greater than or equal to 60×10⁶ cells/mL.

Cell Banking and/or Seed Train or pre-production cell mass increase: In certain embodiments, the cell culture methods disclosed herein may comprise, or may be used for, cell banking. In one aspect, for example, provided herein is a method of cell banking comprising: (a) placing a plurality of mammalian cells and a volume of culture medium in a culture vessel to generate a cell culture; (b) culturing the cell culture to a cell density of greater than or equal to 1% packed cell volume (PCV); (c) conducting a perfusion procedure on the cell culture of step (b), wherein the perfusion procedure comprises: (i) transferring at least a portion of the cell culture to a continuous flow centrifuge; (ii) operating the continuous flow centrifuge to generate a solids phase (heavy phase); (iii) dividing the solids phase into a first portion that is returned to the cell culture vessel, and a second portion, and (iv) returning said first portion to the cell culture vessel (bioreactor); and (d) combining said second portion with a cryopreservative. In a specific embodiment, said second portion is frozen after step (d). In a specific embodiment, the first portion of the solids phase is returned to the cell culture vessel with a volume of cell culture medium. In a more specific embodiment, the return of the first portion of the solids phase and cell culture medium to the cell culture vessel achieves a perfusion rate that ranges from 0.7 to 6 vessel volumes per day (VVD). In a more specific embodiment, following completion of the perfusion procedure, the cell culture has a cell density of 0.2% PCV or greater, e.g., 0.2% to about 30% PCV or greater. In certain embodiments, the cryopreservative is dimethylsulfoxide (DMSO) or growth medium supplemented with DMSO. In specific embodiments, the DMSO has a final concentration of 5% to 10% by volume (vol./vol.). In certain embodiments, the second portion of the heavy phase has a cell density, or is adjusted to a cell density, of at least 1×10⁸ cells per milliliter (cells/mL). In more specific embodiments, the second portion of the heavy phase has a cell density, or is adjusted to a cell density, of at least, about, or no more than 1.0×10⁸ cells/mL, 1.1×10⁸ cells/mL, 1.2×10⁸ cells/mL, 1.3×10⁸ cells/mL, 1.4×10⁸ cells/mL, 1.5×10⁸ cells/mL, 1.6×10⁸ cells/mL, 1.7×10⁸ cells/mL, 1.8×10⁸ cells/mL, 1.9×10⁸ cells/mL, 2.0×10⁸ cells/mL. In certain embodiments, the first portion of the solids phase comprises up to 10%, 15%, 20%, 25%, 30%, 35%, 40%, 45%, or up to 50% of the total volume of the solids phase. In certain embodiments, wherein the first portion of the solids phase comprises up to 10%, 15%, 20%, 25%, 30%, 35%, 40%, 45%, or up to 50% of the total volume of the solids phase, the second portion of the solids phase comprises, or substantially comprises, the remainder of the solids phase. In certain embodiments, the solids phase is divided into said first portion and said second portion multiple discrete times during cell culture. In certain embodiments, the solids phase is divided into said first portion and said second portion one time during cell culture. In certain embodiments, the solids phase is divided into said first portion and said second portion continuously during cell culture. In certain embodiments, the solids phase, or the second portion of the solids phase, has a cell viability of at least 70%, 75%, 80%, 85%, 90%, 91%, 92%, 93%, 94% or 95%. %. In the above embodiments, the centrifuge may be a continuous flow centrifuge, for e.g., a single continuous flow centrifuge. In some embodiments, two or more continuous flow centrifuges is used, e.g., in parallel. In additional embodiments, the cells or cell mass generated from one or more continuous flow centrifuges is used to start or inoculate cultures in one of more pre-production bioreactors, or inoculate or transfer cultures into one or more production bioreactors.

In certain embodiments, the cell banking described above is a part of pre-production cell culture of cells genetically modified to produce a protein of interest, e.g., a therapeutic protein or polypeptide. In certain embodiments, the therapeutic protein or polypeptide is a fusion protein, e.g., a fusion protein comprising an antibody Fc portion or human serum albumin. In other specific embodiments, the therapeutic protein or polypeptide is an antibody or antibody fragment, e.g., a monoclonal antibody, monospecific antibody, bispecific antibody (with or without common light chain), bispecific T cell engager (BiTE), bispecific (mab)₂ antibodies; bispecific F(mab)₂ antibody; single-domain bispecific diabody (scBsDb), single-chain bispecific tandem variable domain (scBsTaFv), trispecific NK cell engager therapy (TriNKET), dual-affinity re-targeting protein (DART), bispecific diabody, tandem diabody (TandAb), half-antibodies (e.g., antibodies comprising an Fc portion but only one CH-VH:CL-VL pair), trifab contorsbody (as described in PCT Application Publication No. WO 2019/086395; quadroma, scFv, dock-and-lock trivalent fab (DNL-(Fab)₃, single-domain antibody, bispecific single-domain antibody, or the like.

### III. SYSTEMS

Various systems both described herein and commercially available may be suitable for carrying out the presently disclosed methods. Systems may be fed-batch and/or continuous flow based. Systems may include agitation systems based on stirred tank or rocking. Such systems may be designed for single-use or reuse. For example, the centrifuge (e.g., a device suitable for inclusion into perfusion methods) may include a disposable component (e.g., a single-use bowl). Examples of fed-batch systems that may also support continuous flow or perfusion techniques for carrying out the methods described herein include, but are not limited to, any one of the HyPerforma™ systems (e.g., HyPerforma™ 5:1 Single-Use Bioreactor, Jacketed, AC motor, Load Cells, Catalog number: SUB00508100) manufactured by Thermo Fisher Scientific™, any one of the Ambr® or Biostat® STR systems (e.g., Biostat STR® Generation 3 featuring Biobrain® Automation) manufactured by Sartorious™, any one of the Allegro™ systems manufactured by Pall®. These systems may be modified or adapted to include perfusion-based equipment and systems. One non-limiting example of a perfusion system may include alternating tangential flow (ATF) filtration (e.g., products sourced from Repligen™) making use of hollow filter fibers. Another non-limiting example of a perfusion system may include tangential flow filtration (e.g., products sourced from Flow Sciences, Inc™). Additional examples of tangential flow filtration (TFF) systems are produced by Sartorious™ (e.g., Sartoflow® 150 Auto Single-Use Tangential Flow Filtration System). Depth filtration may also be used in the perfusion methods described herein. An additional non-limiting example of a perfusion system component may include acoustic settler-based perfusion technology (e.g., products sourced from SonoSep Technologies™). Continuous flow systems may incorporate one or more of the systems described herein as well as other commercially available systems. A variety of centrifugal systems may be included with the fed-batch and perfusion systems described herein to carry out the presently described methods. Preferably, the centrifuge used in the present cell culture methods comprises disposable components that contact the cells and/or cell media, e.g., a disposable bowl or bowl insert assembly. In certain embodiments, the continuous centrifuge comprises a single-use centrifugal pump. A non-limiting example of such a system includes CultureOne™ available from Alfa Laval™ (e.g., an Alfa Laval CultureOne Primo™ single-use cell separator). Other suitable commercial systems include Culturefuge 400 B available from Alfa Laval, GEA Kytero® available from GEA Group Aktiengesellschaft, Ksep® available from Sartorius™, DynaSpin™ from Thermo Fisher, and Unifuge® available from CARR®. In certain embodiments, a single continuous flow centrifuge is used. In other embodiments, two or more continuous flow centrifuges are used, e.g., in parallel. In additional embodiments, the cells or cell mass generated from one or more continuous flow centrifuges is used to start or inoculate cultures in one of more pre-production bioreactors, or inoculate or transfer cultures into one or more production bioreactors.

FIG. 1 is schematic diagram of a system for cell culturing comprising perfusion and a cell separation device (in certain embodiments, a centrifuge; in more specific embodiments, a continuous flow centrifuge) 100 in accordance with one or more embodiments of the cell culture methods provided herein. Generally, a cell culturing system for carrying out the methods described herein involves growing a cell culture in a cell culture vessel 120 by first inoculating a cell culture medium (e.g., Gibco™ CHO perfusion medium) of the cell culture vessel 120 with an appropriate cells (e.g., the cell types described herein, recombinant mammalian cells, recombinant CHO cells, and any other commercially available cell types) from an inoculation vessel 110. The medium comprising the cells may be transferred (e.g., pumped) from the inoculation vessel 110 into the cell culture vessel 120 through a sterile connection 101. An inoculation cell culture (e.g., an inoculation culture of mammalian cells) may have a cell density of greater than or equal to 0.1% PCV. An inoculation cell culture (e.g., an inoculation culture of mammalian cells) may have a cell density of greater than or equal to 1% PCV.

Inoculation preferably is performed under sterile conditions by depositing (e.g., pumping through an aseptic connection) a plurality of mammalian cells comprised within a volume of cell culture medium into a culture vessel to generate a cell culture. In a specific embodiment, inoculation may occur under sterile conditions by depositing (e.g., pumping through an aseptic connection) a plurality of mammalian cells comprised within a volume of cell culture medium in a culture vessel having a working volume of, for example, 80 L to generate a cell culture having a starting cell density of greater than or equal to 1 million cells/mL. In another specific embodiment, inoculation may occur under sterile conditions by depositing (e.g., pumping through an aseptic connection) a plurality of mammalian cells and a volume of cell culture medium in a culture vessel having a working volume of 3,000 L, or 3,600 L, to generate a cell culture having a starting cell density of greater than or equal to 1 million cells/mL.

Once a culture has been expanded to a cell density of greater than or equal to 10% PCV (or alternatively to 1%), at least a portion of the culture may be transferred (e.g., pumped using a peristaltic pump) from a cell culture vessel 120 to a cell separation device 130 through a sterile connection 103.

In specific embodiments of the cell culture systems provided herein, the cell separation device 130 preferably removes a portion of a spent or depleted medium and returns a portion, preferably substantially all, cells back to the cell culture vessel 120. In addition to removing depleted medium from the cell culture vessel 120, fresh medium and/or media is preferably transferred from a fresh fluid vessel 170 through a sterile connection 171 to the cell culture vessel. In such systems, fresh medium may be added back to the cell culture as depleted medium is being removed. In a specific embodiment, cell culture vessel 120 has a working volume, or a total volume, that is greater than or equal to 80 L. In other specific embodiments, cell culture vessel 120 has a working volume, or a total volume, that ranges from 100 L to 3,000 L. In a specific embodiment, cell culture vessel 120 has a working volume, or a total volume, of 100 L. In another specific embodiment, cell culture vessel 120 has a working volume, or a total volume, of 3,000 L. Preferably the process of spent medium removal, cell separation, and return of cell back to cell culture vessel 120 proceeds continuously throughout at least portion of the time the cells are cultured.

In some systems, a cell separation device 130 may include a filtration system (e.g., depth, ATF and/or TFF). In specific embodiments of the cell culture method provided herein, cell separation device 130 includes, or is, a centrifuge. In certain embodiments, cell separation device 130 combines filtration and centrifugation. Prior to or during use, a flush liquid may be used to prime one or more components of the system, e.g., the cell separation device 130. In cases where the cell separation device 130 includes a centrifuge that may be used in a continuous or semi-continuous application, a flush fluid may be used to chase or wash out the portion of cell culture transferred from the cell culture vessel 120 to the cell separation device 130. In certain embodiments, the centrifuge is a single continuous flow centrifuge. In an aspect of such embodiments, two or more continuous flow centrifuges are used, e.g., in parallel. In additional embodiments, the cells or cell mass generated from one or more continuous flow centrifuges is used to start or inoculate cultures in one of more pre-production bioreactors, or inoculate or transfer cultures into one or more production bioreactors.

The flush fluid may include a buffer and/or purified water. The buffer may be suitable for maintaining healthy cells (e.g., a phosphate buffered saline (PBS) solution at an appropriate pH so that cells are less likely to lyse). The flush fluid may be pumped from a flush liquid vessel 160 to the cell separation device 130 through a sterile connection 111. In many embodiments, the flush fluid may be used to empty the cell separation device 130 to preserve cells and/or product. In some systems, the flush fluid may be used to dislodge solids from walls of cell separation device 130, e.g., a centrifuge.

Some traditional centrifugal systems include outlets for a light phase and a heavy phase. In such traditional systems, solids (e.g., the cells being cultured) may collect in a bowl of the centrifuge and either need to be discharged (intermittently or consciously) or scraped out periodically. Such procedures interrupt continuous flow. While such systems may be incorporated into the cell culture methods provided herein (e.g., as cell separation device 130), the cell culture methods preferably incorporate a continuous flow cell separator, e.g., a continuous flow centrifuge.

In various systems described herein, a heavy phase may include such solids (e.g., a solids phase comprising cultured cells). A light phase (e.g., a liquid phase) may include a product (e.g., a secreted product from a cell, a recombinant protein, and/or an antibody). A centrifugal system (e.g., a disc-stack centrifuge, tubular centrifuge) used to carry out the methods described herein may be used to separate a liquid phase from a solids phase. The solids phase may include cells. In many systems, solids may not be ejected from the centrifugal system, as may be the case in traditional systems, but may be retained in a solids phase.

In various systems, a cell separation device 130 may include a liquid phase outlet 112. In various embodiments, the liquid phase outlet 112 may include a sterile connection 109 leading to a liquid phase collection vessel 150. The liquid phase being transferred to the liquid phase collection vessel 150 may include one or more products from the cell culture. Once isolated, a purification procedure may be performed on the product (e.g., a product secreted from the cells).

Optionally, a portion of the liquid phase may be transferred back to the cell culture vessel 120 through a sterile connection 113.

In some systems, a liquid phase outlet 112 may include a heavy liquid phase outlet and a light liquid phase outlet. In such systems, the heavy liquid phase outlet may transfer cells back to the cell culture vessel 120 through a sterile connection 113 and the light liquid phase outlet may include depleted medium.

In various systems, a cell separation device 130 may include a solids phase outlet 106. In many systems, the solids phase outlet 106 may transfer all or a portion of a solids phase back to a cell culture vessel 120 through a sterile connection 105. The solids phase may include densely packed cells. In some cases, the solids phase may comprise a highly viscous solution. The centrifuges described herein may operate continuously to generate a solids phase having a cell density ranging from 1% to 50% PCV, e.g., about 10%, about 10%, about 30%, about 40% or about 50% PCV. The centrifuges described herein may operate continuously to generate a solids phase having a cell density of greater than or equal to 30% PCV.

One or more pumps (e.g., centrifugal pump or peristaltic pump) may return at least a portion of the solids phase and a volume of cell culture medium to the cell culture vessel 120 to achieve a perfusion rate that ranges from 0.5 to 6 vessel volumes per day (VVD), wherein following completion of the perfusion procedure, the cell culture has a cell density of 0.2% PCV or greater. Additionally, a fresh cell culture fluid may be pumped into the cell culture vessel 120 from a fresh cell culture medium vessel 170 through a sterile connection 171 to replace the depleted medium.

One or more pumps (e.g., centrifugal pump or peristaltic pump) may return at least a portion of the solids phase and a volume of cell culture medium to the culture vessel 120 to achieve a perfusion rate that ranges from 0.5 to 6 VVD, wherein following completion of the perfusion procedure, the cell culture has a cell density of greater than or equal to 0.1% PCV. Additionally, a fresh cell culture fluid may be pumped into the cell culture vessel 120 from a fresh cell culture medium vessel 170 through a sterile connection 171 to replace the depleted medium.

One or more pumps (e.g., centrifugal pump or peristaltic pump) may return at least a portion of the solids phase and a volume of cell culture medium to the culture vessel 120 to achieve a perfusion rate that ranges from 0.5 to 6 VVD, wherein following completion of the perfusion procedure, the cell culture has a cell density of greater than or equal to 60×10⁶ cells/ml. Additionally, a fresh cell culture fluid may be pumped into the cell culture vessel 120 from a fresh cell culture medium vessel 170 through a sterile connection 171 to replace the depleted medium.

One or more pumps (e.g., centrifugal pump or peristaltic pump) may return at least a portion of the solids phase and a volume of cell culture medium to the cell culture vessel 120 to achieve a perfusion rate that ranges from 2 to 6 VVD. Additionally, a fresh cell culture fluid may be pumped into the cell culture vessel 120 from a fresh cell culture medium vessel 170 through a sterile connection 171 to replace the depleted medium.

The one or more pumps may operate to increase or decrease the perfusion rate in a constant manner. The one or more pumps may operate to increase or decrease the perfusion rate in a variable manner. The one or more pumps may operate continuously over a time period that ranges from 1 to 7 days. The one or more pumps may operate in a semi-continuous manner over a time period that ranges from 1 to 7 days.

A centrifugal process may result in a cell culture having a viability percentage that is greater than or equal to 85%. A centrifugal process may result in a cell culture that maintains a viability percentage that is greater than or equal to 85% over the time period of 1 to 7 days. A centrifugal process may result in a cell culture having a lactate concentration that is less than or equal to 4 g/L.

The centrifuge may operate at a speed that ranges from 3,000 to 10,000 RPM. A continuous flow centrifuge may have a sigma value that ranges from 1,000 m² to 200,000 m².

A cell separation device 130 may include a sterilizable component. For example, a cell separation device may be hermetically sealed. When the cell separation device 130 includes a centrifuge bowl (e.g., disc stack or tubular), rotating components (e.g., a drive shaft) may include one or more seals. In some cases, a sterilization component may include a heating component. Additionally, one or more aseptic connectors may couple the continuous flow centrifuge to tubing or lines directly or indirectly coupled to a cell culture vessel 120, a flush liquid vessel, 160, one or more solids phase collection vessels 140, and/or a liquid phase collection vessel 150.

In some systems, a solids phase outlet 106 may transfer all or a portion of a solids phase to a solids phase collection vessel 140 through a sterile connection 107. In some systems, the solids phase collection vessel 140 may be a cell culture vessel. In some systems, the solids phase collection vessel 140 may be a plurality of cell culture vessels. The solids phase may be used to inoculate a medium of one or more solids phase collection vessels 140. For example, the system may transfer at least a portion of the cell culture to a different culture vessel to initiate a second cell culture. The second cell culture may have an initial cell density that ranges from 0.1% to 10% PCV.

In some systems, the cell culture vessel 120 may include a production culture vessel. The system may transfer at least a portion of the cell culture to a production culture vessel to initiate a production culture having a starting cell density that ranges from 0.1% to 10% PCV. The production culture undergo continuous processing conditions. The production culture undergo batch or fed-batch processing conditions.

The sterile connections 101, 103, 105, 107, 109, 111, 113 described herein may include tubing connected to the vessels and devices 110, 120, 130, 140, 150, 160 through one or more aseptic connectors and may be pumped using one or more pumps (e.g., peristaltic pumps). Tubing, pumps, and aseptic connectors are commercially available from a variety of manufacturers, including Thermo Fisher Scientific™, Sartorious™, and Pall®.

Environmental conditions, including but not limited to, cell density, cell viability, pH, dO₂, pressure, and temperature may be monitored within the cell culture vessel 120 or in any other component of the system by one or more sensors or probes. In automated cell culturing processes, the one or more sensors or probes may send a signal to a control system reporting the environmental conditions. The control system may then act to adjust one or more of the environmental conditions by activating pumps, heating elements, or any ancillary device capable for changing the environmental conditions.

### IV. METHODS OF USE

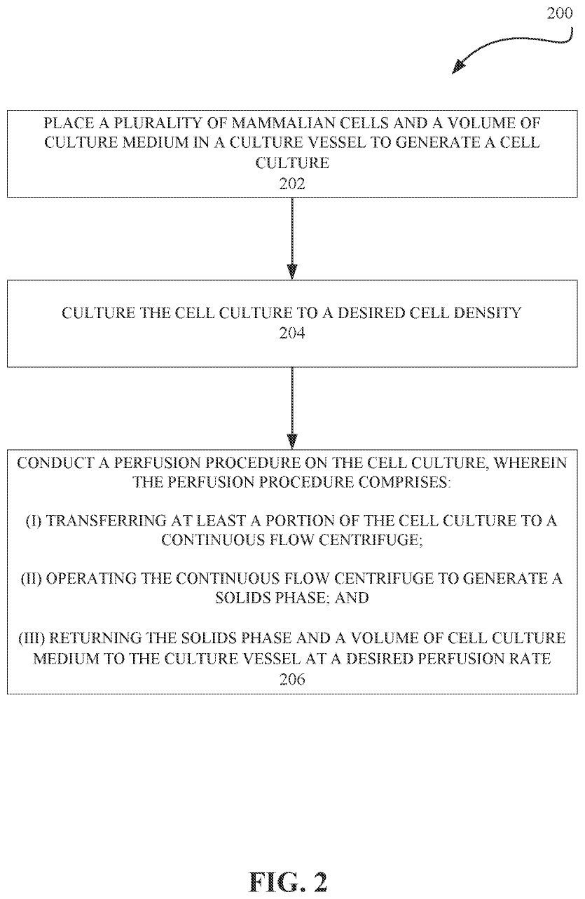

FIG. 2 is a flowchart of a process for culturing mammalian cells 200 in accordance with one or more embodiments.

Step 1 includes placing a plurality of mammalian cells and a volume of culture medium in a culture vessel to generate a cell culture 202.

Step 2 includes culturing the cell culture to a desired cell density 204.

Step 3 includes conducting a perfusion procedure on the cell culture 206.

A perfusion procedure may include transferring at least a portion of the cell culture to a continuous flow centrifuge. A perfusion procedure may include operating the continuous flow centrifuge to generate a solids phase. A perfusion procedure may include returning the solids phase and a volume of cell culture medium to the culture vessel at a desired perfusion rate.

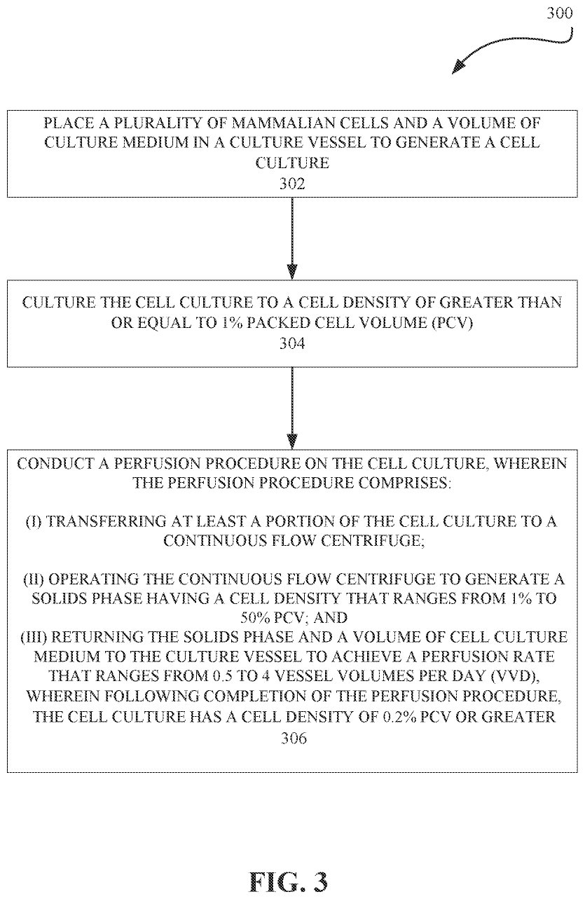

FIG. 3 is a flowchart of a process for culturing mammalian cells 300 in accordance with one or more embodiments.

Step 1 includes placing a plurality of mammalian cells and a volume of culture medium in a culture vessel to generate a cell culture 302. In some cultures, the mammalian cells may include recombinant mammalian cells. The recombinant mammalian cells may include recombinant Chinese hamster ovary (CHO) cells. The recombinant mammalian cells may produce a secreted product. In various processes, the secreted product may include a recombinant protein. In various processes, the recombinant protein may include an antibody.

Various cultures may use a culture vessel that has a working volume that may be greater than or equal to 80 L. Various cultures may use a culture vessel that has a total volume that may range from 100 L to 3,000 L. Some cultures may use a vessel having a total volume of 100 L. Some cultures may use a vessel having a total volume of 3,000 L.

Step 2 includes culturing the cell culture to a cell density of greater than or equal to 1% packed cell volume (PCV) 304.

Step 3 includes conducting a perfusion procedure on the cell culture 306.

Conducting a perfusion procedure may include returning a solids phase and a volume of cell culture medium to the culture vessel to achieve a perfusion rate that ranges from 0.5 to 6 vessel volumes per day (VVD). In some perfusion procedures, the perfusion rate ranges from 2 to 4 VVD. Some perfusion procedures may include increasing or decreasing the perfusion rate in a constant manner. Alternative perfusion procedures may include increasing or decreasing the perfusion rate in a variable manner.

A perfusion procedure may be conducted continuously over a time period that ranges from 1 to 7 days. Alternative profusion procedures may be conducted in a semi-continuous manner over a time period that ranges from 1 to 7 days. The cell culture may maintain a viability percentage that is greater than or equal to 85% over the time period of 1 to 7 days.

Conducting a perfusion procedure may include transferring at least a portion of the cell culture to a continuous flow centrifuge. Some perfusion procedures may use a continuous flow centrifuge that includes a disc stack bowl. The continuous flow centrifuge may have a sigma value that ranges from 1,000 m² to 200,000 m². The continuous flow centrifuge may maintain a residence time temperature of between about 30° C. and about 39° C., e.g., between 31° C. and 38° C., between 32° C. and 38° C., between 33° C. and 38° C., between 34° C. and 38° C., between 35° C. and 38° C. or between 36° C. and 38° C.

Alternatively perfusion procedures may include use of a continuous flow centrifuge that includes a tubular bowl. A variety of suitable centrifugal systems are commercially available.

Conducting a perfusion procedure may include operating a continuous flow centrifuge to generate a solids phase having a final cell density of greater than or equal to 1% PCV, e.g., about 10%, about 20%, about 30%, about 40% or about 50% PCV.

Conducting a perfusion procedure may include isolating a liquid phase from the continuous flow centrifuge and performing a purification procedure on a secreted product therein.

The continuous flow centrifuges used in the perfusion procedures described herein may include a sterilizable component. A sterilization component may include one or more devices or processes described herein and elsewhere.

The continuous flow centrifuges used in the perfusion procedures described herein may include a disposable component.

Continuous flow centrifuges used in a profusion procedure may include an operating speed that ranges from 3,000 to 10,000 RPM.

In some process for culturing mammalian cells, culturing the production culture under batch or fed-batch process conditions.

Following completion of the centrifugation procedure of the perfusion procedure, a cell culture may have a viability percentage that is greater than or equal to 85%. Following completion of the centrifugation procedure of the perfusion procedure, the cell culture may have a lactate concentration that is less than or equal to 4 g/L. Following completion of the perfusion procedure, the cell culture may have a cell density of 0.2% PCV or greater, e.g., 0.2% PCV to about 30% PCV.

A process for culturing mammalian cells may include transferring at least a portion of the cell culture to a different culture vessel to initiate a second cell culture. In some processes, the second cell culture may have an initial cell density that ranges from 0.1% to 10% PCV.

A process for culturing mammalian cells may include transferring at least a portion of the cell culture to a production culture vessel to initiate a production culture having a starting cell density that ranges from 0.1% to 10% PCV.

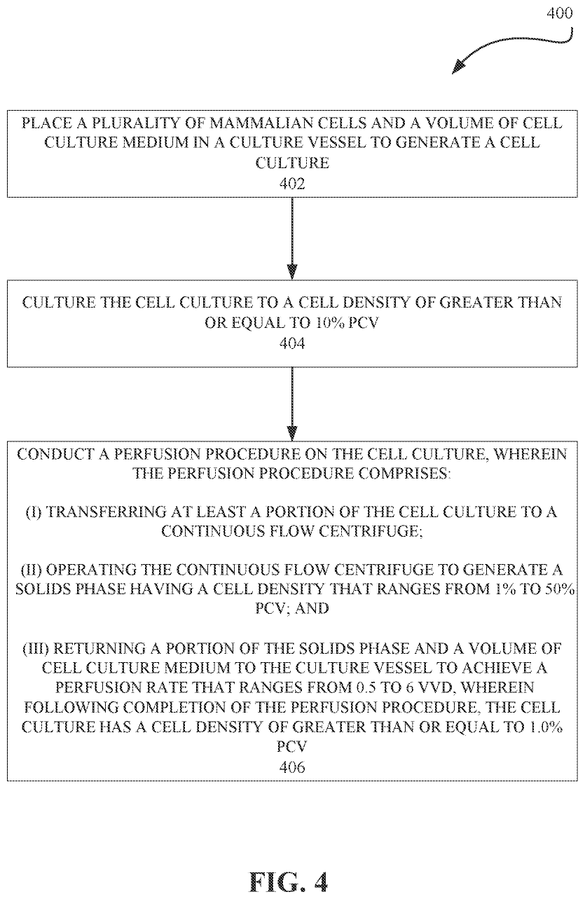

FIG. 4 is a flowchart of a process for generating an inoculation culture of mammalian cells having a cell density of greater than or equal to 0.10% PCV 400 in accordance with one or more embodiments, e.g., 0.1% to about 30% PCV.

Step 1 includes placing a plurality of mammalian cells and a volume of cell culture medium in a culture vessel to generate a cell culture 402.

Step 2 includes culturing the cell culture to a cell density of greater than or equal to 10% PCV 404.

Step 3 includes conducting a perfusion procedure on the cell culture 406.

A perfusion procedure may include transferring at least a portion of the cell culture to a continuous flow centrifuge. The perfusion procedure may include operating the continuous flow centrifuge to generate a solids phase having a cell density that ranges from 1% to about 50% PCV. The perfusion procedure may include returning a portion of the solids phase and a volume of cell culture medium to the culture vessel to achieve a perfusion rate that ranges from 0.5 to 6 VVD, wherein following completion of the perfusion procedure, the cell culture has a cell density of greater than or equal to 0.10% PCV to 30% PCV.

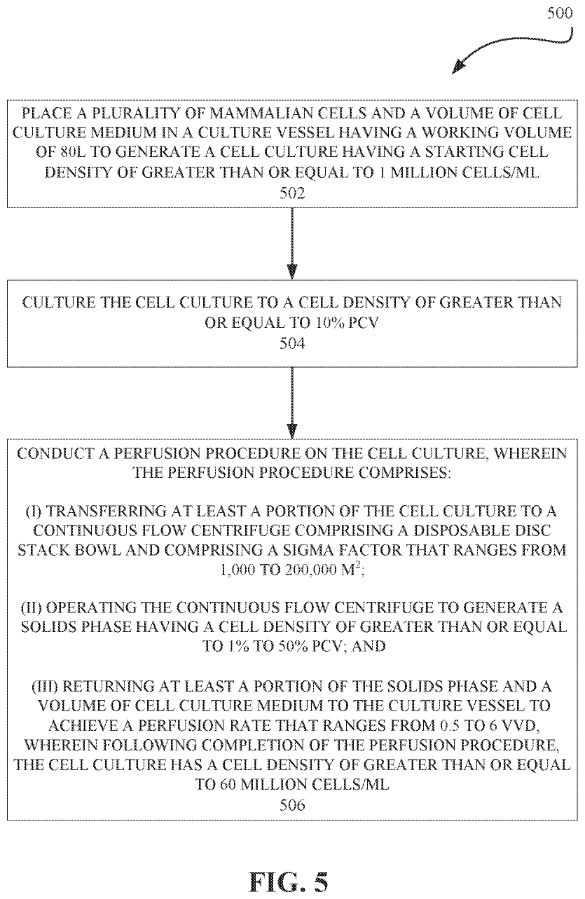

FIG. 5 is a flowchart of a process for generating an inoculation culture of mammalian cells comprising at least 4.8×10¹² cells 500 in accordance with one or more embodiments.

Step 1 includes placing a plurality of mammalian cells and a volume of cell culture medium in a culture vessel having a working volume of 80 L to generate a cell culture having a starting cell density of greater than or equal to 1 million cells/mL 502.

Step 2 includes culturing the cell culture to a cell density of greater than or equal to 10% PCV 504.

Step 3 includes conducting a perfusion procedure on the cell culture 506.

A perfusion reaction may include transferring at least a portion of the cell culture to a continuous flow centrifuge comprising a disposable disc stack bowl and comprising a sigma factor that ranges from 1,000 to 200,000 m². The perfusion procedure may include operating the continuous flow centrifuge to generate a solids phase having a final cell density of greater than or equal to 1% PCV, e.g., about 10%, about 20%, about 30%, about 40% or about 50% PCV. The perfusion procedure may include returning at least a portion of the solids phase and a volume of cell culture medium to the culture vessel to achieve a perfusion rate that ranges from 0.5 to 6 VVD, wherein following completion of the perfusion procedure, the cell culture has a cell density of greater than or equal to 60 million cells/mL.

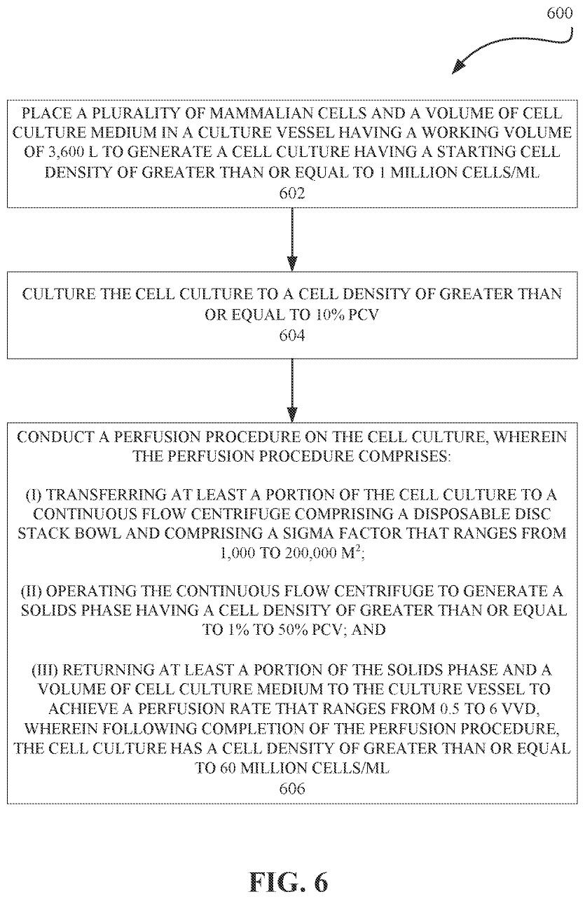

FIG. 6 is a flowchart of a process for generating an inoculation culture of mammalian cells comprising at least 2.16×10¹⁴ cells 600 in accordance with one or more embodiments.

Step 1 includes placing a plurality of mammalian cells and a volume of cell culture medium in a culture vessel having a working volume of 3,000 L, or 3,600 L to generate a cell culture having a starting cell density of greater than or equal to 1 million cells/mL 602.

Step 2 includes culturing the cell culture to a cell density of greater than or equal to 10% PCV 604.

Step 3 includes conducting a perfusion procedure on the cell culture 606.

A perfusion procedure may include transferring at least a portion of the cell culture to a continuous flow centrifuge comprising a disposable disc stack bowl and comprising a sigma factor that ranges from 1,000 to 200,000 m². The perfusion procedure may include operating the continuous flow centrifuge to generate a solids phase having a final cell density of greater than or equal to 1% PCV, e.g., about 10%, about 20%, about 30%, about 40% or about 50% PCV. The perfusion procedure may include returning at least a portion of the solids phase and a volume of cell culture medium to the culture vessel to achieve a perfusion rate that ranges from 0.7 to 6 VVD, wherein following completion of the perfusion procedure, the cell culture has a cell density of greater than or equal to 60 million cells/mL.

The processes in methods described herein may be carried out using the systems and devices described herein and elsewhere.

For the technologies described herein, it will be apparent to one of ordinary skill in the art that various changes and modifications can be made without departing from the spirit or scope of the present embodiments.

### EXAMPLES

### Example 1: 50% Split Ratio

A perfusion cell culture run where a ratio of solids phase to liquid phase of 50% was returned to the production cell culture was completed. The centrifuge was adjusted to volumetrically flow 50% of the centrifuge inlet flow to the solids outlet and 50% of the centrifuge inlet flow through the liquid outlet for a split ratio of 50:50 solids to liquid.

### Example 2: 75% Split Ratio

A perfusion cell culture run where a ratio of solids phase to liquid phase of 75% is returned to the production cell culture is completed. The centrifuge is adjusted to volumetrically flow 75% of the centrifuge inlet flow to the solids outlet and 25% of the centrifuge inlet flow through the liquid outlet for a split ratio of 75:25 solids to liquid.

### Example 3: 90% Split Ratio

A perfusion cell culture run where a ratio of solids phase to liquid phase of 90% is returned to the production cell culture is completed. The centrifuge is adjusted to volumetrically flow 90% of the centrifuge inlet flow to the solids outlet and 10% of the centrifuge inlet flow through the liquid outlet for a split ratio of 90:10 solids to liquid.

### Example 4: Seven-Day Cell Culture

This Example demonstrates a seven-day cell culture incorporating the present continuous centrifuge cell culture method, without a clarification step in preparation for protein harvesting.

Mammalian cells (Chinese Hamster Ovary (CHO) cells) that are capable of expressing a therapeutic antibody were grown in a bioreactor with a 500 liter working volume using a target actual volume of approximately 300 L. The packed cell volume (PCV) at the start of cell culture was 0.30% with a viable cell count (VCC) of 2.087×10⁶ cells/mL at a viability of 97.7%. After culturing for ˜19 hours, the PCV rose to 0.40% with a VCC of 3.472×10⁶ cells/mL at a viability of 97.9%. At about 67 hours culture, a perfusion procedure was initiated using an Alfa Laval CultureOne Primo™ continuous centrifuge at 2.5 vessel volumes per 24 hour period (VVD), which resulted in a perfusion flow rate of 521 mL/min. The centrifuge was operated with a target split ratio of 50% (that is, the solids phase and light phase was 50:50) and an actual centrifuge inlet flow rate of 1040 mL/min. The solids phase was returned to the cell culture vessel and the light phase was discarded for this run). The advantage of this continuous centrifuge is that the bowl insert assembly (Spinsert™) is designed as a disposable single-use assembly that simplifies maintenance of sterility, and provides gentle handling of cells (promoting higher cell viability). The centrifuge was supplemented with peristaltic pumps connected to the inlet and outlet ports. Chilled water (about 4° C. to 10° C.) was used to cool the centrifuge seals and minimize the temperature gains from centrifuge spinning Medium addition throughout the perfusion process was added at a flow rate equal to the liquid phase flow rate to maintain the bioreactor actual volume, controlled by a bioreactor load cell The bowl speed was increased from 3000 RPM to 3200 RPM to 3400 RPM to optimize cell retention. At this point, the bioreactor PCV was about 2.2%, with a VCC of 1.24×10⁷ cells/mL and viability of 97.10%. Cell culture perfusion procedure continued for the next ˜46.5 hours, and consequently the bioreactor PCV rose to 4.33%, and the VCC rose to about 2.5×10⁷ cells/mL with a viability of 91.7%.

At about 113.5 hours, the perfusion flow rate was increased from 2.5 VVD to 3.7 VVD (equivalent to a target flow rate of 771 mL/min. at the 300 L working volume) with an actual centrifuge inlet flow rate of 1540 mL/min. Five hours later, bioreactor PCV was about 4.8%, cell viability was at 92.8% with a VCC of 3.069×10⁷ cells/mL. Over the next 22.5 hours the PCV rose to about 7.40%, and the VCC rose to about 4.038×10⁷ cells/mL, with a viability of 92.6%.

At approximately 137.5 hours post-initiation, the perfusion flow rate was increased from 3.7 VVD to 3.96 VVD (for reference, 3.96 VVD was equivalent to a target flow rate of 825 mL/min. at the 300 L working volume) with an actual centrifuge inlet flow rate of 1650 mL/min. The centrifuge continued to operate with a target split ratio of 50%. The cell viability was at 93.8% after the change. Over the next 27 hours, the PCV rose to about 12.0%, cell viability was at 95.1%, and the VCC rose to about 5.66×10⁷ cells/mL.

At approximately 164.5 hours post-initiation, the perfusion flow rate was increased from 3.96 VVD to 5 VVD (for reference, 5 VVD was equivalent to a target flow rate of 1042.0 mL/min at the 300 L working volume). The centrifuge continued to operate with a target split ratio of 50%, with an actual centrifuge inlet flow rate of 2080 mL/min. The solids phase flow rate was 1040 mL/min, which required a manual peristaltic pump to be installed at this point. Bioreactor cell viability was at 95.9% after this change. Cell culture with centrifugation was continued until about 181.5 hours after culture initiation. Starting at 3.96 VVD, the solids phase was at 14% PCV; at the end of the run at 5 VVD, the solids phase achieved 32% PCV. Cell viability in the culture vessel was about 93.2% at this point, with a PCV of about 16% and a VCC of about 8.435×10⁷ cells/mL, while running at 5 VVD. From when the solids phase was first sampled, starting at about 3.96 VVD and at about 14% PCV, by the end of the run, at about 5 VVD the packed cell volume increased to about 32% PCV.

At every point in time during this culture-centrifugation process, bioreactor cell viability was at least 91.4%, and at higher perfusion rates (above 3.7 VVD), bioreactor cell viability was generally above 92%.

While preferred embodiments of the present technologies have been shown and described herein, it will be obvious to those skilled in the art that such embodiments are provided by way of example only. Numerous variations, changes, and substitutions may now occur to those skilled in the art without departing from the described technologies. It should be understood that various alternatives to the embodiments described herein may be employed in practicing the systems and methods. It is intended that the following claims cover, not only the described methods and structures, but also their equivalents.

1. A method for culturing mammalian cells, comprising:
(a) placing a plurality of mammalian cells and a volume of culture medium in a culture vessel to generate a cell culture;
(b) culturing the cell culture to a cell density of greater than or equal to 1% packed cell volume (PCV);
(c) conducting a perfusion procedure on the cell culture during step (b), wherein the perfusion procedure comprises:
(i) transferring at least a portion of the cell culture to a continuous flow centrifuge;
(ii) operating the continuous flow centrifuge to generate a solids phase having a cell density of greater than or equal to 1% PCV; and
(iii) returning the solids phase and a volume of cell culture medium to the culture vessel to achieve a perfusion rate that ranges from 0.7 to 6 vessel volumes per day (VVD), wherein following completion of the perfusion procedure, the cell culture has a cell density of 0.2% PCV or greater.

2. The method of claim 1, wherein the perfusion rate ranges from 2 to 6 VVD.

3. The method of claim 1, wherein the perfusion procedure comprises increasing or decreasing the perfusion rate in a constant manner.

4. The method of claim 1, wherein the perfusion procedure comprises increasing or decreasing the perfusion rate in a variable manner.

5. The method of claim 1, wherein the perfusion procedure is conducted continuously or semicontinuously over a time period that ranges from 1 to 7 days.

6. (canceled)

7. The method of claim 1, wherein the continuous flow centrifuge comprises a disc stack bowl.

8. The method of claim 1, wherein the continuous flow centrifuge comprises a tubular bowl.

9. The method of claim 1, wherein the continuous flow centrifuge has an operating speed that ranges from 3,000 to 10,000 RPM.

10.-12. (canceled)

13. The method of claim 1, wherein following completion of the centrifugation procedure, the cell culture has a viability percentage that is greater than or equal to 85%.

14. The method of claim 5, wherein the cell culture maintains a viability percentage that is greater than or equal to 85% over the time period of 1 to 7 days.

15. The method of claim 1, wherein the mammalian cells comprise recombinant mammalian cells.

16. The method of claim 15, wherein the recombinant mammalian cells comprise recombinant Chinese hamster ovary (CHO) cells.

17. The method of claim 16, wherein following completion of the centrifugation procedure, the cell culture has a lactate concentration that is less than or equal to 4 g/L.

18. The method of claim 15, wherein the recombinant mammalian cells produce a secreted product.

19. The method of claim 15, wherein the secreted product comprises a recombinant protein.

20. The method of claim 19, wherein the recombinant protein is an antibody.

21. (canceled)

22. The method of claim 1, wherein the culture vessel has a total volume that ranges from 100 L to 30,000 L.

23. (canceled)

24. (canceled)

25. The method of claim 1, further comprising transferring at least a portion of the cell culture to a different culture vessel to initiate a second cell culture.

26. The method of claim 25, wherein the second cell culture has an initial cell density that ranges from 0.1% to 10% PCV.

27. The method of claim 1, further comprising transferring at least a portion of the cell culture to a production culture vessel to initiate a production culture having a starting cell density that ranges from 0.1% to 10% PCV.

28.-30. (canceled)

31. A method of generating a culture of mammalian cells having a cell density of greater than or equal to 0.1% PCV, the method comprising:
(a) placing a plurality of mammalian cells and a volume of cell culture medium in a culture vessel to generate a cell culture;
(b) culturing the cell culture to a cell density of greater than or equal to 10% PCV;
(c) conducting a perfusion procedure on the cell culture during step (b), wherein the perfusion procedure comprises:
(i) transferring at least a portion of the cell culture to a continuous flow centrifuge;
(ii) operating the continuous flow centrifuge to generate a solids phase having a cell density that ranges from about 1% to about 50% PCV; and
(iii) returning a portion of the solids phase and a volume of cell culture medium to the culture vessel to achieve a perfusion rate that ranges from 0.7 to 6 VVD, wherein following completion of the perfusion procedure, the cell culture has a cell density of greater than or equal to 0.1% PCV.

32. A method of generating a culture of mammalian cells comprising at least 4.8×10¹² cells, the method comprising:
(a) placing a plurality of mammalian cells and a volume of cell culture medium in a culture vessel having a working volume of 80 L to generate a cell culture having a starting cell density of greater than or equal to 1 million cells/mL;
(b) culturing the cell culture to a cell density of greater than or equal to 10% PCV;
(c) conducting a perfusion procedure on the cell culture during step (b), wherein the perfusion procedure comprises:
(i) transferring at least a portion of the cell culture to a continuous flow centrifuge comprising a disposable disc stack bowl and comprising a sigma factor that ranges from 1,000 to 200,000 m²;
(ii) operating the continuous flow centrifuge to generate a solids phase having a cell density of greater than or equal to 1% PCV; and
(iii) returning at least a portion of the solids phase and a volume of cell culture medium to the culture vessel to achieve a perfusion rate that ranges from 0.7 to 6 VVD, wherein following completion of the perfusion procedure, the cell culture has a cell density of greater than or equal to 60 million cells/mL.

33. A method of generating a culture of mammalian cells comprising at least 2.16×10¹⁴ cells, the method comprising:
(a) placing a plurality of mammalian cells and a volume of cell culture medium in a culture vessel having a working volume of 3,000 L to generate a cell culture having a starting cell density of greater than or equal to 1 million cells/mL;
(b) culturing the cell culture to a cell density of greater than or equal to 10% PCV;
(c) conducting a perfusion procedure on the cell culture of step (b), wherein the perfusion procedure comprises:
(i) transferring at least a portion of the cell culture to a continuous flow centrifuge comprising a disposable disc stack bowl and comprising a sigma factor that ranges from 1,000 to 200,000 m²;
(ii) operating the continuous flow centrifuge to generate a solids phase having a cell density of greater than or equal to 1% PCV; and
(iii) returning at least a portion of the solids phase and a volume of cell culture medium to the culture vessel to achieve a perfusion rate that ranges from 0.7 to 6 VVD, wherein following completion of the perfusion procedure, the cell culture has a cell density of greater than or equal to 60 million cells/mL.

34. A method of cell banking comprising:
(a) placing a plurality of mammalian cells and a volume of culture medium in a culture vessel to generate a cell culture;
(b) culturing the cell culture to a cell density of greater than or equal to 1% packed cell volume (PCV);
(c) conducting a perfusion procedure on the cell culture of step (b), wherein the perfusion procedure comprises:
(i) transferring at least a portion of the cell culture to a continuous flow centrifuge;
(ii) operating the continuous flow centrifuge to generate a solids phase (heavy phase);
(iii) dividing the solids phase into a first portion that is returned to the cell culture vessel, and a second portion, and
(iv) returning said first portion to the cell culture chamber;
(d) combining said second portion with a cryopreservative.

35.-50. (canceled)
